SEQUENCE LISTING (13 sequences)

SEQ ID NO: 1 - protein, 9 aa, Artificial Sequence
  Synthetic Peptide
RRRRRRRRR

SEQ ID NO: 2 - protein, 13 aa, Artificial Sequence
  Synthetic Peptide
GRKKRRQRRRPPQ

SEQ ID NO: 3 - protein, 22 aa, Artificial Sequence
  Synthetic Peptide
MVTVLFRRLRIRRACGPPRVRV

SEQ ID NO: 4 - protein, 16 aa, Artificial Sequence
  Synthetic Peptide
RQIKIWFQNRRMKWKK

SEQ ID NO: 5 - protein, 21 aa, Artificial Sequence
  Synthetic Peptide
AGYLLGKINLKALAALAKKIL

SEQ ID NO: 6 - protein, 27 aa, Artificial Sequence
  Synthetic Peptide
GALFLGFLGAAGSTMGAWSQPKKKRKV

SEQ ID NO: 7 - protein, 30 aa, Artificial Sequence
  Synthetic Peptide
WEAKLAKALAKALAKHLAKALAKALKACEA

SEQ ID NO: 8 - protein, 20 aa, Artificial Sequence
  Synthetic Peptide
GLFKALLKLLKSLWKLLLKA

SEQ ID NO: 9 - protein, 27 aa, Artificial Sequence
  Synthetic Peptide
GALFLGFLGAAGSTMGAWSQPKSKRKV

SEQ ID NO: 10 - protein, 27 aa, Artificial Sequence
  Synthetic Peptide
GALFLAFLAAALSLMGLWSQPKKKRKV

SEQ ID NO: 11 - protein, 9 aa, Artificial Sequence
  Synthetic Peptide
RRRRRRRRR

SEQ ID NO: 12 - protein, 20 aa, Artificial Sequence
  Synthetic Peptide
GLWRALWRLLRSLWRLLWRA

SEQ ID NO: 13 - protein, 14 aa, Artificial Sequence
  Synthetic Peptide
VSRRRRRRGGRRRR

(19) United States
(12) Patent Application Publication
(10) Pub. No.: US 2018/0291086 A1
(43) Pub. Date: Oct. 11, 2018

(54) LIPOPLEXES FORMULATED FOR CATALYTIC DELIVERY

(71) Applicant: THE REGENTS OF THE UNIVERSITY OF COLORADO, a body corporate, Denver, CO (US)

(72) Inventors: Thomas ANCHORDOQUY, Lakewood, CO (US); Jamie L. BETKER, Denver, CO (US)

(73) Assignee: The Regents of the University of Colorado, a body corporate, Denver, CO (US)

(21) Appl. No.: 15/766,088

(22) PCT Filed: Oct. 5, 2016
(86) PCT No.: PCT/US2016/055570

§ 371 Date: Apr. 5, 2018

Related U.S. Application Data
(60) Provisional application No. 62/237,473, filed on Oct. 5, 2015.

(51) Int. Cl. C07K 14/775; A61K 9/107; A61K 47/28; A61K 9/127; C07J 9/00

(52) U.S. Cl. CPC C07K 14/775; A61K 9/107; C07J 9/00; A61K 9/1272; A61K 47/28

(57) ABSTRACT

Lipoplex formulations and uses thereof. In particular, the lipoplexes include at least one naturally-occurring cationic amphiphile, at least one C18-30 saturated fatty acid, cholesterol, at least one nucleic acid, and have a low charge ratio. The lipoplexes are useful for in vivo or in vitro delivery of one or more agents (e.g., a polyanionic therapeutic or an antisense therapeutic, such as an RNAi agent) and allow prolonged expression of these agents, which may be distributed via endogenous cellular pathways to surrounding cells or tissue.

### GOVERNMENT INTEREST

This invention was made with U.S. government support under grant numbers 1RO1GM093287 and GM093287 awarded by the National Institutes of Health (NIH). The U.S. Government has certain rights in this invention.

### TECHNICAL FIELD

The invention relates to lipoplexes, as well as formulations thereof, and their use in the delivery of therapeutic agents, such as nucleic acid molecules, to cells.

### BACKGROUND OF DISCLOSURE

It is well-established that the delivery of nucleic acids to the interior of the target cell represents a major barrier to the therapeutic use of genes and RNA. Successful delivery systems for nucleic acids must exhibit stability in the blood, accumulation at the target site, uptake and efficient trafficking within the target cell. It is generally accepted that accumulation in the tumor is primarily governed by the enhanced permeation and retention effect, whereby leaky tumor vasculature allows particles of sufficiently small size to extravasate into the tumor, at least in animal models. Studies have demonstrated that the mobility of nanoparticles is restricted in the extracellular tumor environment, and the use of ligands enhances the uptake of targeted nanoparticles by the tumor cells.

Accordingly, there is a need for new formulations for the delivery of therapeutic agents, such as RNAi agents. In particular, lipid formulations capable of delivering nucleic acid therapeutics to cells.

### SUMMARY OF INVENTION

This disclosure provides novel lipoplex formulations for the delivery of one or more therapeutic agents. In particular, these lipoplex formulations can be used to deliver a polyanionic therapeutic or an antisense therapeutic (e.g., nucleic acid molecules or RNAi agents) to cells to induce a desired effect, such as silence a target gene.

The lipoplexes of this disclosure possess several unique characteristics that result in dramatic improvements over, conventional delivery systems.

Based on previous studies of liposomes and lipoplex fromulations, it is recognized that the delivery vehicle itself can impart toxicity, and this is especially pertinent because the delivery of nucleic acids is dependent on cationic agents that are known to be cytotoxic. Although biodegradable, monovalent cationic agents are better tolerated, but the toxicity of gene delivery vehicles is still a significant concern, as organ toxicity can limit dosing and ultimately reduce therapeutic efficacy. Similarly, uptake of a lipoplex delivery vehicle and its associated cationic agents can compromise the viability of transfected cells. While these effects are not typically observed in an overnight, in vitro transfection experiment, recent studies have shown that even a brief exposure of cells to nucleic acids complexed with standard transfection reagents elicits a toxic response that slowly progresses to cell death over a week. The loss of viability results in a progressive decrease in reporter gene expression that limits the duration for which therapeutic genes are expressed in vivo. Thus, the development of less toxic delivery vehicles of this disclosure enables transfected cells to maintain their viability; thereby allowing therapeutic expression to persist for extended timeframes. With respect to cancer therapies, these extended timeframes of expression may be sufficient to ensure that immune suppression by the tumor is reversed. The use of naturally-occurring cationic amphiphiles to serve as the cationic component in the lipoplex delivery systems of this disclosure leads to lower toxicity, better compatibility, and extended expression of expression constructs delivered by these lipoplex vehicles for prolonged periods.

Because expression constructs (i.e., plasmids) that expresses antisense or other nucleic acid therapeutics may be effectively delivered using the lipoplex formulations of this disclosure, it will be important to maximize delivery and retention at the delivery site (e.g., at the tumor for cancer therapies). Although non-targeted lipoplex particles of this disclosure are capable of delivering nucleic acid therapeutics at levels resulting in robust tumor expression, the additional incorporation of a targeting ligand in these lipoplexes can increase levels of gene expression in the target tissues. But simply incorporating a ligand into a particle does not necessarily enhance uptake or specificity. In fact, studies have shown that the proteins adsorbed to a nanoparticle after systemic administration can obscure/foul targeting ligands (Hagiwara K, Ochiya T, Kosaka N. A paradigm shift for extracellular vesicles as small RNA carriers: from cellular waste elimination to therapeutic applications. Drug Deliv Transl Re. 2014; 4:31-7.) An approach to avoid ligand fouling that has been extensively studied is the use of PEGylated components. But concerns about PEG immunogenicity and reduced intracellular delivery suggest that superior delivery might be achieved by avoiding the use of PEG in delivery formulations. Indeed, multiple studies have documented that even very low levels of PEGylation 1%) can significantly reduce transfection rates (Lee J, et al Liposome-Based Engineering of Cells To Package Hydrophobic Compounds in Membrane Vesicles for Tumor Penetration. Nano letters. 2015; 15:2938-44; Syn N, et al. Exosome-Mediated Metastasis: From Epithelial-Mesenchymal Transition to Escape from Immunosurveillance. Trends Pharmacol Sci. 2016; 37:606-17; Smyth T, et al. Biodistribution and delivery efficiency of unmodified tumor-derived exosomes. Journal of Controlled Release. 2015; 199:145-55). Thus, avoiding PEGylated components is another distinct advantage of the lipoplex delivery systems of this disclosure. But the use of targeting ligands necessitates the use of an effective linker to link the chosen ligand(s) to the lipoplex components, such as cholesterol. The lipoplex delivery systems of this disclosure possess a cholesterol domain. This aspect of these lipoplexes imparts a distinct advantage in that undetectable amounts of protein are adsorbed to these domains, making them ideal for presenting targeting ligands. Furthermore, conjugating a targeting ligand to cholesterol preferentially locates the ligand within the protein-free cholesterol domain, which enhances the transfection rates of the lipoplexes of this disclosure, both in vitro and in vivo. Cholesterol membrane domains formed within lipoplexes of this disclosure endow these lipoplexes with improved serum stability, transfection, and targeting both in vitro and in vivo. The inventors have conclusively demonstrated that the formation of a cholesterol domain is responsible for these beneficial effects. Additionally, the cholesterol domain presents a region on the lipoplex that does not adsorb measurable levels of serum proteins, and therefore is ideal for presenting targeting ligands. Fortunately, specific localization of targeting ligands within the domain can be readily achieved by using cholesterol as an anchor.

In addition to the reduced toxicity of the delivery vehicle, use of a cholesterol nanodomain for improved delivery/ligand presentation, and exploitation of endogenous cellular pathways (such as the exosomal pathway) to enhance therapeutic nucleic acid distribution and expression within the tumor, the lipoplex formulations of this disclosure may employ expression constructs capable of extended expression to maximize therapeutic protein/antisense therapeutic levels in target tissues (such as tumor cells). The use of such expression constructs capable of extended expression results in robust expression that may extend for at least 10 days. Additional modifications may be employed to limit the duration of expression and/or incorporate inducible promoters, if desired.

The combination of minimal vehicle toxicity with extended expression results in yet another advantageous characteristic of the lipoplexes of this disclosure: repetitive dosing of these lipoplexes is possible, thereby achieving significantly higher expression of the therapeutic nucleic acid due to the cumulative effects of multiple target tissue transfections. The inventors have demonstrated this effect quite clearly, showing that it can take days for some plasmids delivered to tumors via the lipoplex formulations of this disclosure to initiate expression. This delayed expression presumably results from deferred uptake at the plasma membrane and/or nucleus, and culminates in a dramatic increase in expression resulting from the combined effects of multiple doses. In these experiments on tumor tissues, the transgene expression was primarily confined to the tumor, thus limiting systemic effects.

The delivery of therapeutic nucleic acids (e.g., siRNA, aptmers, encoded antigen) resulting in expression of these nucleic acids in the target tissue offers a distinct therapeutic advantage because these nucleic acids are known to be transferred within tumor tissue, via exogenous pathways, including the exosomal pathway. Thus, delivery of such nucleic acid constructs via the administration of lipoplexes of this disclosure can achieve local amplification of the delivered nucleic acid expression construct (e.g., an siRNA-mediated silencing expression construct), and the inventors have demonstrated that exosomes harvested from transfected cells contain the siRNA encoded by the plasmid. Prolonged expression in the fraction of tumor cells accessed by the lipoplex delivery systems of this disclosure allows distribution of the therapeutic nucleic acid (e.g., siRNA) throughout the target tissue via the exosomal pathway, creating a robust bystander effect which is further enhanced by the prolonged expression of these constructs that extends for days or even weeks beyond the initial lipoplex administration.

In one aspect, the lipoplexes of this disclosure are formulated with expression constructs that express siRNA known to locally inhibit the expression of ZEB1 or PD-L1, a protein that plays a critical role in the ability of tumors to evade the immune system. By distribution of these PD-L1-silencing constructs within tumors via the endogenous pathways and the prolonged expression of the siRNA construct within a fraction of the target cancer cells throughout the tumor mass, the inventors have demonstrated a reduction in tumor expression of this endogenous tumor target gene by 95%. Similarly, by expressing microRNA200c at high levels in a tumor, expression of the downstream target of the microRNA (ZEB1) was reduced by five orders of magnitude. Thus, the expression of these gene targets were essentially knocked out in the transfected cells. This unprecedented level of gene silencing indicated that expression in all cells associated with the tumor was inhibited, confirming that the RNA responsible for silencing must have been widely distributed throughout the tumor.

Exploitation of this endogenous distribution system within tumors can be combined with the targeted delivery of lipoplexes of this disclosure to enhance target tissue uptake while simultaneously dramatically reducing toxicity, allowing repetitive administration without a refractory period between injections, resulting in superior delivery to tumors (accumulating additional multi-fold expression after repeated injections). Thus, the combined advantages of the lipoplexes of this disclosure yield dramatic improvements over conventional delivery systems.

In one aspect, this disclosure provides a lipoplex formulation including: at least one cationic amphiphile, C₁₈₋₃₀ saturated fatty acids, and at least one nucleic acid. These lipoplexes may also include cholesterol. A portion of the cholesterol optionally may be conjugated to a ligand that promotes uptake from the vasculature into target cells, e.g., uptake into the tumor vasculature or into cancer cells.

These lipoplexes may be formulated to have a charge ratio between 0.1 and 20.0. The charge ratio is the mole ratio of cations (in the delivery vehicle) to anions (typically phosphates in the nucleic acid). These lipoplexes may be formulated to have a charge ratio between 0.25 and 4.0. These lipoplexes may also be formulated to have a charge ratio between 0.25 and 1.0.

Cholesterol may be included in these lipoplexes. If present, the lipoplexes may include a cholesterol content greater than 5% by weight, including greater than 10% by weight, including greater than 20% by weight. The cholesterol content of these lipoplexes may be between about 10% by weight and about 30% by weight.

The cholesterol optionally may also include a ligand conjugated directly to all or a portion of the cholesterol. The ligand may be iRGD peptide (CRGDKGPDC). The ligand conjugation may be substantially- or completely-free of PEG linkers. The ligand conjugation may be substantially- or completely-free of PEG linkers. The ligand conjugation may be an ester linkage to cholesterol present in these lipoplexes. Such ester linkage to cholesterol can be subsequently cleaved by cholesterol esterase, which may further enhance distribution of these lipoplexes in tumors.

Ligands useful in the lipoplexes of this disclosure bind to cancer cells or the relevant tissue or organ. These ligands may specifically bind to a marker expressed on cancer cells or a marker up-regulated on cancer cells compared to normal cells. The ligand may specifically bind to a cancer-specific antigen (e.g., CEA (carcinoembryonic antigen) (colon, breast, lung); PSA (prostate specific antigen) (prostate cancer); CA-125 (ovarian cancer); CA 15-3 (breast cancer); CA 19-9 (breast cancer); HER2/neu (breast cancer); α-feto protein (testicular cancer, hepatic cancer); 13-HCG (human chorionic gonadotropin) (testicular cancer, choriocarcinoma); MUC-1 (breast cancer); estrogen receptor (breast cancer, uterine cancer); progesterone receptor (breast cancer, uterine cancer); EGFR (epidermal growth factor receptor) (bladder cancer)), and folate. Thus, depending on the ligand(s) incorporated into the lipoplexes of this disclosure, these lipoplexes are useful in the treatment of melanoma, colon, breast, lung, prostate, uterine, and bladder cancers.

The ligand may be any type of compound including, without limitation, peptides, proteins, antibodies (e.g., monoclonal antibodies, antibody fragments, antibody mimics, etc.), lipids, glycoproteins, carbohydrates, small molecules, and derivatives and combinations thereof. The ligand may be an RGD peptide or RGD mimic/analog (see, e.g., European Patent Application EP2239329; U.S. Patent Publication No. 2010/0280098). The RGD peptide may be, without limitation, a cyclic RGD (cRGD) or internalizing RGD (iRGD). The RGD peptides may also be a monomer or dimer. The ligand may include a cell penetrating peptide, such as polyarginines (RRRRRRRRR; SEQ ID NO:1), TAT (GRKKRRQRRRPPQ; SEQ ID NO:2), M918 (MVTVLFRRLRIRRACGPPRVRV; SEQ ID NO:3), Penetratin (RQIKIWFQNRRMKWKK; SEQ ID NO:4), TP10 (AGYLLGKINLKALAALAKKIL; SEQ ID NO:5), MPG (GALFLGFLGAAGSTMGAWSQPKKKRKV; SEQ ID NO:6), KALA (WEAKLAKALAKALAKHLAKALAKALKACEA; SEQ ID NO:7), ppTG1 (GLFKALLKLLKSLWKLLLKA; SEQ ID NO:8), MPGΔNLS (GALFLGFLGAAGSTMGAWSQPKSKRKV; SEQ ID NO:9), MPGα (GALFLAFLAAALSLMGLWSQPKKKRKV; SEQ ID NO:10), Chol-R9 (Cholesteryl-RRRRRRRRR; SEQ ID NO:11), CADY (Ac-GLWRALWRLLRSLWRLLWRA (SEQ ID NO:12)-cysteamide), and/or LMWP (VSRRRRRRGGRRRR; SEQ ID NO:13).

The lipoplexes may have a C₁₈₋₃₀ saturated fatty acid content greater than 15% by weight, including greater than 20% by weight, and greater than 50% by weight. The C₁₈₋₃₀ saturated fatty acid content of these lipoplexes may be between about 20% by weight and about 60% by weight. Saturated fatty acids used in these lipoplexes may be predominately C₁₈₋₂₄ saturated fatty acids.

The cationic amphiphile content of these lipoplexes may include one or more naturally-occurring cationic amphiphile, such as sphingosine, sphinganine, dimethylsphingosine, phytosphingosine, and stearylamine. The lipoplexes may have a cationic amphiphile content greater than 1% by weight, including greater than 5% by weight, including greater than 15% by weight. The cationic amphiphile content of these lipoplexes may be between about 5% by weight and about 30% by weight.

The formulation may further include a cationic lipid (e.g., DODMA, DOTMA, DPePC, DODAP, or DOTAP), a neutral lipid (e.g., DSPC, POPC, DOPE, or SM), and, another sterol derivative (e.g., cholestanone; cholestenone; coprostanol; 3β-[-(N—(N′,N′-dimethylaminoethane)-carbamoyl]cholesterol (DC-cholesterol); bis-guanidium-tren-cholesterol (BGTC); (2S,3S)-2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenant-hren-3-yloxy)carbonylamino)ethyl 2,3,4,4-tetrahydroxybutanoate (DPC-1); (2S,3S)-((3S,10R,13R,17R)-10,13-dimethyl-17-((R)-6-methylheptan-2-yl)-2,3-,4,7,8,9,10,11,12,13,14,15,16,17-tetradecahydro-1H-cyclopenta[a]phenanthre-n-3-yl) 2,3,4,4-tetrahydroxybutanoate (DPC-2); bis((3S,10R,13R,17R)-10,13-dimethyl-17-((R)-6-methylheptan-2-yl)-2,3,4,7,-8,9,10,11,12,13,14,15,16,17-tetradecahydro-1H-cyclopenta[a]phenanthren-3-yl) 2,3,4-trihydroxypentanedioate (DPC-3); or 6-(((3S,10R,13R,17R)-10,13-dimethyl-17-((R)-6-methylheptan-2-yl)-2,3,4,7,-8,9,10,11,12,13,14,15,16,17-tetradecahydro-1H-cyclopenta[a]phenanthren-3-yloxy)oxidophosphoryloxy)-2,3,4,5-tetrahydroxyhexanoate (DPC-4)).

The formulation may include from about 10 mol % to about 80 mol % (e.g., from about 40 mol % to about 55 mol %) of one or more C₁₈₋₃₀ saturated fatty acids, from about 1 mol % to about 70 mol % of one or more C₁₈₋₃₀ saturated fatty acids.

These lipoplex formulations may include from about 1 mol % to about 80 mol % (e.g., from about 40 mol % to about 55 mol %) of one or more sterol, from about 1 mol % to about 70 mol % of one or more sterol.

These lipoplex formulations further include a polyanionic therapeutic or an antisense therapeutic. The polyanionic therapeutic may be an RNAi agent (e.g., dsRNA, siRNA, miRNA, shRNA, ptgsRNA, or DsiRNA, e.g., DsiRNA). The RNAi agent may have a length of 10 to 40 nucleotides, e.g., length of 10 to 15 nucleotides, 10 to 20 nucleotides, 10 to 25 nucleotides, 10 to 30 nucleotides, 10 to 35 nucleotides, 15 to 20 nucleotides, 15 to 25 nucleotides, 15 to 30 nucleotides, 15 to 35 nucleotides, 15 to 40 nucleotides, 16 to 20 nucleotides, 16 to 25 nucleotides, 16 to 30 nucleotides, 16 to 35 nucleotides, 16 to 40 nucleotides, 20 to 25 nucleotides, 18 to 20 nucleotides, 18 to 25 nucleotides, 18 to 30 nucleotides, 18 to 35 nucleotides, 18 to 40 nucleotides, 19 to 20 nucleotides, 19 to 25 nucleotides, 19 to 30 nucleotides, 19 to 35 nucleotides, 19 to 40 nucleotides, 20 to 30 nucleotides, 20 to 35 nucleotides, 20 to 40 nucleotides, 25 to 30 nucleotides, 25 to 35 nucleotides, 25 to 40 nucleotides, 30 to 35 nucleotides, 30 to 40 nucleotides, or 35 to 40 nucleotides, e.g., a length of 25 to 35 nucleotides, e.g., a length of 16 to 30 nucleotides, e.g., a length of 19 to 29 nucleotides. The antisense therapeutic may have a length of 8 to 50 nucleotides (e.g., a length of 8 to 10 nucleotides, 8 to 15 nucleotides, 8 to 15 nucleotides, 8 to 20 nucleotides, 8 to 25 nucleotides, 8 to 30 nucleotides, 8 to 35 nucleotides, 8 to 40 nucleotides, or 8 to 45 nucleotides), e.g., a length of 14 to 35 nucleotides (e.g., a length of 14 to 15 nucleotides, 14 to 20 nucleotides, 14 to 25 nucleotides, or 14 to 30 nucleotides), e.g., a length of 17 to 24 nucleotides, e.g., a length of 17 to 20 nucleotides.

The polyanionic therapeutic may be a nucleic acid based antigen (i.e., a RNA or DNA-vaccine construct for systemic targeting of immune system cells and synchronized induction of both highly potent adaptive as well as type-I-interferon-mediated innate immune mechanisms for cancer immunotherapy). In this instance, the polyanionic therapeutic may have a nucleotide length between 10 and several thousand nucleotides. The polyanionic therapeutic may be an aptmer.

The polyanionic therapeutic may be a plasmid, or other expression construct, that encodes one or more of microRNAs, siRNAs, shRNAs, antigens, aptmers, and the like, resulting in the expression of the encoded peptides/proteins following administration and cellular uptake of the lipoplexes. The polyanionic therapeutic may be a polynucleotide that encodes proteins that can be shuttled/distributed via the cellular exosomal pathway.

The formulation may include from about 1:10 (w/w) to about 1:100 (w/w) ratio of the polyanionic therapeutic to the total lipid present in the formulation, e.g., from about 1:10 (w/w) to about 1:15 (w/w) ratio, from about 1:10 (w/w) to about 1:20 (w/w) ratio, from about 1:10 (w/w) to about 1:40 (w/w) ratio, from about 1:10 (w/w) to about 1:50 (w/w) ratio, from about 1:10 (w/w) to about 1:60 (w/w) ratio, from about 1:10 (w/w) to about 1:70 (w/w) ratio, from about 1:10 (w/w) to about 1:80 (w/w) ratio, from about 1:10 (w/w) to about 1:90 (w/w) ratio, from about 1:10 (w/w) to about 1:95 (w/w) ratio, from about 1:20 (w/w) to about 1:40 (w/w) ratio, from about 1:20 (w/w) to about 1:50 (w/w) ratio, from about 1:20 (w/w) to about 1:60 (w/w) ratio, from about 1:20 (w/w) to about 1:70 (w/w) ratio, from about 1:20 (w/w) to about 1:80 (w/w) ratio, from about 1:20 (w/w) to about 1:90 (w/w) ratio, from about 1:20 (w/w) to about 1:95 (w/w) ratio, from about 1:20 (w/w) to about 1:100 (w/w) ratio, from about 1:40 (w/w) to about 1:50 (w/w) ratio, from about 1:40 (w/w) to about 1:60 (w/w) ratio, from about 1:40 (w/w) to about 1:70 (w/w) ratio, from about 1:40 (w/w) to about 1:80 (w/w) ratio, from about 1:40 (w/w) to about 1:90 (w/w) ratio, from about 1:40 (w/w) to about 1:95 (w/w) ratio, from about 1:40 (w/w) to about 1:100 (w/w) ratio, from about 1:50 (w/w) to about 1:60 (w/w) ratio, from about 1:50 (w/w) to about 1:70 (w/w) ratio, from about 1:50 (w/w) to about 1:80 (w/w) ratio, from about 1:50 (w/w) to about 1:90 (w/w) ratio, from about 1:50 (w/w) to about 1:95 (w/w) ratio, from about 1:50 (w/w) to about 1:100 (w/w) ratio, from about 1:60 (w/w) to about 1:70 (w/w) ratio, from about 1:60 (w/w) to about 1:80 (w/w) ratio, from about 1:60 (w/w) to about 1:90 (w/w) ratio, from about 1:60 (w/w) to about 1:95 (w/w) ratio, from about 1:60 (w/w) to about 1:100 (w/w) ratio, from about 1:80 (w/w) to about 1:90 (w/w) ratio, from about 1:80 (w/w) to about 1:95 (w/w) ratio, or from about 1:80 (w/w) to about 1:100 (w/w) ratio of the polyanionic therapeutic to the total lipid present in the formulation.

In one aspect, this disclosure provides a pharmaceutical composition including any lipoplex formulation described herein and a pharmaceutically acceptable excipient.

In another aspect, this disclosure provides a method of treating or prophylactically treating a disease in a subject, the method including administering to the subject any lipoplex or pharmaceutical composition described herein in an amount sufficient to treat the disease or disorder. The disease may be a genetic disease or disorder. The disease may be cancer (e.g., liver cancer or other neoplastic diseases and associated complications including, but not limited to, carcinomas (e.g., lung, breast, pancreatic, colon, hepatocellular, renal, female genital tract, prostate, squamous cell, carcinoma in situ), lymphoma (e.g., histiocytic lymphoma, non-Hodgkin's lymphoma), MEN2 syndromes, neurofibromatosis (including Schwann cell neoplasia), myelodysplastic syndrome, leukemia, tumor angiogenesis, cancers of the thyroid, liver, bone, skin, brain, central nervous system, pancreas, lung (e.g., small cell lung cancer, non small cell lung cancer), breast, colon, bladder, prostate, gastrointestinal tract, endometrium, fallopian tube, testes and ovary, gastrointestinal stromal tumors (GISTs), prostate tumors, mast cell tumors (including canine mast cell tumors), acute myeloid myelofibrosis, leukemia, acute lymphocytic leukemia, chronic myeloid leukemia, chronic lymphocytic leukemia, multiple myeloma, melanoma, mastocytosis, gliomas, glioblastoma, astrocytoma, neuroblastoma, sarcomas (e.g., sarcomas of neuroectodermal origin or leiomyosarcoma), and metastasis of tumors to other tissues.

In another aspect, this disclosure provides a method of modulating the expression of a target nucleic acid in a subject, the method including administering any lipoplex or pharmaceutical composition described herein in an amount sufficient to reduce the expression of the target gene.

In these methods, the administration of a lipoplex or pharmaceutical composition of this disclosure to a subject may occur one or more times per day (e.g., 1, 2, 3, or 4 times per day), one or more times per week (e.g., 2, 3, 4, 5, 6, or 7 times per week) or one or more times per month (e.g., 2, 3, 4, 5, 6, 7, or 10 times per month). A subject may receive dosages of these lipoplexes in the range of about 0.001 to about 200 mg/kg, e.g., about 0.001 to about 1 mg/kg, about 0.001 to about 10 mg/kg, about 0.001 to about 20 mg/kg, about 0.001 to about 50 mg/kg, about 0.001 to about 100 mg/kg, about 0.01 to about 1 mg/kg, about 0.01 to about 10 mg/kg, about 0.01 to about 20 mg/kg, about 0.01 to about 50 mg/kg, about 0.01 to about 100 mg/kg, about 0.01 to about 200 mg/kg, about 0.1 to about 1 mg/kg, about 0.1 to about 10 mg/kg, about 0.1 to about 20 mg/kg, about 0.1 to about 50 mg/kg, about 0.1 to about 100 mg/kg, about 0.1 to about 200 mg/kg, about 1 to about 10 mg/kg, about 1 to about 20 mg/kg, about 1 to about 50 mg/kg, about 1 to about 100 mg/kg, about 1 to about 200 mg/kg, about 10 to about 20 mg/kg, about 10 to about 50 mg/kg, about 10 to about 100 mg/kg, about 10 to about 200 mg/kg, about 20 to about 50 mg/kg, about 20 to about 100 mg/kg, or about 20 to about 200 mg/kg, in any dosage regimen (e.g., one or more times per day (e.g., 1, 2, 3, or 4 times per day), one or more times per week (e.g., 2, 3, 4, 5, 6, or 7 times per week) or one or more times per month (e.g., 2, 3, 4, 5, 6, 7, or 10 times per month)). In exemplary embodiments, the administration of a lipoplex or pharmaceutical composition of this disclosure to a subject may occur between every 24 to 72 hours, preferably about every 72 hours (i.e., every 3 days).

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### Definitions

As used herein, the term “about” means +/−10% of the recited value.

As used herein, the term “immunocompetent” refers to animal models containing a fully functioning/intact immune system.

As used herein, the term “gene delivery:” means the delivery of nucleic acids to cells in culture or in vivo.

As used herein, the term “biodistribution” means the distribution of administered material within a living organism, usually focusing on major organs, e.g., liver, lungs, kidney, spleen, heart, tumor.

A ‘4T1’ cell is a cell derived from a mouse mammary carcinoma that can be used in immunocompetent mice.

As used herein, the term “liver toxicity” is the toxicity of administered material in the liver; typically assessed by monitoring elevation in specific liver enzymes, e.g., alanine aminotransferase (ALT).

As used herein, the term “charge ratio” means the molar ratio of cationic charges provided by the delivery vehicle to anionic charges on the polyanionic therapeutic.

As used herein, the term “cholesterol nanodomain” refers to the phenomenon in which, under certain conditions, cholesterol will form phase-separated regions within a membrane that possess distinctly different physicochemical properties. Previous studies have shown that the presence of such domains greatly enhances the stability and transfection activity of lipoplexes.

By “linker” is meant an optionally substituted polyvalent (e.g., divalent) group containing one or more atoms. Examples of linkers include esters linkages to cholesterol molecules present in the lipoplexes of this disclosure. Linkers preferably are substantially or completely free of PEG or derivatives thereof.

By “microRNA” (miRNA) is meant a single-stranded RNA molecule that can be used to silence a gene product through RNA interference. These RNA molecules may have complementary sequences that allow them to fold like tRNA.

By “polyanionic therapeutic” is meant a chemical moiety comprising multiple negatively charged atoms that may be incorporated into a lipoplex of this disclosure. Examples of a polyanionic therapeutic include nucleic acids, RNAi agents, siRNA, dsRNA, miRNA, shRNA, DsiRNA, antisense therapeutics, and DNA- or RNA-vaccine constructs.

By “RNA-binding agent” is meant any agent or combination of agents capable of binding or hybridizing a nucleic acid, e.g., a nucleic acid therapeutic of a therapeutic formulation. RNA-binding agents include any lipid described herein (e.g., one or more cationic lipids, combinations of one or more cationic lipids, such as those described herein or in Table 1, as well as combinations of one or more cationic lipids and any other lipid, such as neutral lipids or PEG-lipid conjugates). The RNA-binding agent can form any useful structure within a formulation, such as an internal aggregate.

By “RNAi agent” is meant any agent or compound that exerts a gene silencing effect by hybridizing a target nucleic acid. RNAi agents include any nucleic acid molecules that are capable of mediating sequence-specific RNAi (e.g., under stringent conditions), for example, a short interfering RNA (siRNA), double-stranded RNA (dsRNA), microRNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and Dicer-substrate RNA (DsiRNA).

By “short hairpin RNA” or “shRNA” is meant a sequence of RNA that makes a tight hairpin turn and is capable of gene silencing.

By “silencing” or “gene silencing” is meant that the expression of a gene or the level of an RNA molecule that encodes one or more proteins is reduced in the presence of an RNAi agent below that observed under control conditions (e.g., in the absence of the RNAi agent or in the presence of an inactive or attenuated molecule such as an RNAi molecule with a scrambled sequence or with mismatches). Gene silencing may decrease gene product expression by 1%, 2%, 3%, 4%, 5%, 6%, 7%, 8%, 9%, 10%, 15%, 20%, 25%, 30%, 35%, 40%, 50%, 60%, 70%, 80%, 85%, 90%, 95%, 97%, 98%, 99%, or 100% (i.e., complete inhibition).

By “small inhibitory RNA,” “short interfering RNA,” or “siRNA” is meant a class of 10-40 (e.g., 15-25, such as 21) nucleotide double-stranded molecules that are capable of gene silencing. Most notably, siRNA are typically involved in the RNA interference (RNAi) pathway by which the siRNA interferes with the expression of a specific gene product.

By “pharmaceutical composition” is meant a composition containing a lipoplex of this disclosure formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

By “pharmaceutically acceptable excipient” is meant any ingredient other than the lipoplexes described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

By “subject” or “patient” is meant either a human or non-human animal (e.g., a mammal).

As used herein, “treatment” or “treating” is an approach for obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment. By “treating cancer,” “preventing cancer,” or “inhibiting cancer” is meant causing a reduction in the size of a tumor or the number of cancer cells, slowing or inhibiting an increase in the size of a tumor or cancer cell proliferation, increasing the disease-free survival time between the disappearance of a tumor or other cancer and its reappearance, preventing or reducing the likelihood of an initial or subsequent occurrence of a tumor or other cancer, or reducing an adverse symptom associated with a tumor or other cancer. In a desired embodiment, the percent of tumor or cancerous cells surviving the treatment is at least 20, 40, 60, 80, or 100% lower than the initial number of tumor or cancerous cells, as measured using any standard assay. Desirably, the decrease in the number of tumor or cancerous cells induced by administration of a lipoplex of this disclosure is at least 2, 5, 10, 20, or 50-fold greater than the decrease in the number of non-tumor or non-cancerous cells. Desirably, the methods of this disclosure result in a decrease of 20, 40, 60, 80, or 100% in the size of a tumor or number of cancerous cells, as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after no less than 5, 10, 15, or 20 years. By “prophylactically treating” a disease or condition (e.g., cancer) in a subject is meant reducing the risk of developing (i.e., the incidence) of or reducing the severity of the disease or condition prior to the appearance of disease symptoms. The prophylactic treatment may completely prevent or reduce appears of the disease or a symptom thereof and/or may be therapeutic in terms of a partial or complete cure for a disease and/or adverse effect attributable to the disease. Prophylactic treatment may include reducing or preventing a disease or condition (e.g., preventing cancer) from occurring in an individual who may be predisposed to the disease but has not yet been diagnosed as having the disease or disorder.

### BRIEF DESCRIPTION OF DRAWINGS

FIG. 1 shows expression following lipoplex administration in vitro. Lipoplexes formulated with a constant amount of DNA at different charge ratios were used to transfect 4T1 cells. Luciferase expression was monitored after 48 h, and symbols represent the mean and standard error of multiple wells (n=8). Closed circles—4 Chol:1 DOTAP; Open circles—4 Chol:1 Sphingosine; Closed triangles—2 Chol:3 DOTAP:5 DAPC; Open triangles—2 Chol:3 Spingosine:5 DAPC; Closed squares—1 Chol:1 DOTAP; Open squares—1 Chol:1 Sphingosine; Closed diamonds—Lipofectamine.

FIG. 2 shows expression following lipoplex administration in vitro. Lipoplexes formulated with constant cationic lipid at different charge ratios were used to transfect 4T1 cells. Luciferase expression was monitored after 48 h, and symbols represent the mean and standard error of multiple wells (n=8). Closed circles—4 Chol:1 DOTAP; Open circles—4 Chol:1 Sphingosine; Closed triangles—2 Chol:3 DOTAP:5 DAPC; Open triangles—2 Chol:3 Spingosine:5 DAPC; Closed squares—1 Chol:1 DOTAP; Open squares—1 Chol:1 Sphingosine; Closed diamonds—Lipofectamine.

FIGS. 3A and 3B show the blood levels of lipoplexes in Balb/c mice bearing 4T1 tumors. qPCR was used to determine DNA levels over 6 h. Lipoplexes formulated at +/−=4 (FIG. 3A) and +/−=0.5 (FIG. 3B). Closed circles—lipofectamine; Open Circles—4 Chol:1 DOTAP; Closed Triangles—4 Chol:1 Sphingosine; Open Triangles—2 Chol:3 DOTAP:5 DAPC; Closed squares—2 Chol:3 Sphiingosine:5 DAPC. Symbols represent the mean and standard error of samples from 3 mice.

FIGS. 4A and 4B show DNA levels (FIG. 3A) and expression (FIG. 3B) in tumors after 24 h. Balb/c mice bearing 4T1 tumors were administered lipoplexes formulated at different charge ratios; +/−=4 (black bars), +/−=0.5 (gray bars). Bars represent the mean and standard error; n=6.

FIG. 5 shows the biodistribution of plasmid in different organs. Lipoplexes formulated at +/−=4 (black bars) or +/−=0.5 (gray bars) were administered to Balb/c mice bearing 4T1 tumors. Organs were harvested 24 h after injection, and plasmid levels determined by qPCR. Bars represent the mean and standard error; n=3.

FIG. 6 shows the reporter gene expression in different organs. Lipoplexes formulated at +/−=4 (black bars) or +/−=0.5 (gray bars) were administered to Balb/c mice bearing 4T1 tumors. Organs were harvested 24 h after injection, and luciferase activities determined. Bars represent the mean and standard error; n=3.

FIG. 7 shows liver toxicity following lipoplex administration in vivo. Lipoplexes formulated at +/−=4 (black bars) or +/−=0.5 (gray bars) were administered to Balb/c mice bearing 4T1 tumors. Blood was collected 24 h after injection, and ALT levels determined. Bars represent the mean and standard error; n=3.

FIG. 8 shows lipoplex clearance from plasma following IV administration to mice. Plasma was collected from mice 5, 30, 60, 240 and 1440 minutes after each intravenous administration of lipoplexes, and plasmid was subsequently quantified by qPCR. Symbols and error bars represent the mean and one standard error of samples taken from three mice.

FIGS. 9A and 9B show quantification of plasmid levels (FIG. 9A) and luciferase expression (FIG. 9B) in tumors extracted from mice 24 h after the first and fourth administration of lipoplexes. The bars represent the mean and one standard error of six tumors extracted from three mice. Asterisks indicate statistically significant differences (p<0.0001).

FIGS. 10A and 10B show quantification of plasmid levels (FIG. 10A) and luciferase expression (FIG. 10B) in tissues extracted from mice 24 h after the first and fourth administration of lipoplexes. The bars represent the mean and one standard error of tissues extracted from three mice. Note log scale in B. Asterisks indicate statistically significant differences (p<0.05).

FIG. 11 shows blood levels of ALT measured after intravenous injection of lipoplexes (dark bars) or PBS (light bars). The bars represent the mean and one standard error of ALT levels in blood extracted at each timepoint from three mice. Asterisks indicate statistically significant differences (p<0.004).

FIG. 12 shows luciferase activity imaged in mice 24 and 72 h after repetitive injections of lipoplexes. Each row of images was taken from an individual mouse at the indicated times.

FIG. 13 shows that the lipid formulation of the lipoplexes of this disclosure elicits minimal cytokine response.

FIG. 14 shows the delivery of plasmid encoding miRNA 200c to mice bearing ovarian cancer tumors.

FIG. 15 shows the in vivo expression of shRNAs against immune inhibitory receptor PD-L1.

FIGS. 16A and 16B shows exosomes isolated from human ovarian cancer cells (FIG. 16A) or murine colon carcinoma cells (FIG. 16B) and transfected with plasmid encoding RNAs to silence ZEB1 (FIG. 16A) or PD-L1 (FIG. 16B). RNA levels in the exosomes were measured via qPCR.

FIGS. 17A and 17B show in vivo silencing of ZEB1 in ovarian cancer (FIG. 17A) or PD-L1 in murine carcinoma (CT26, FIG. 17B) tumors 10 days and 4 days, respectively, after delivery of a plasmid encoding a silencing RNA.

### DESCRIPTION OF EMBODIMENTS

The present disclosure provides lipoplex formulations that may be used for the delivery of a polyanionic therapeutic (e.g., nucleic acid molecules or RNAi agents) to cells (e.g., in vitro or in vivo in a subject). The delivery of the polyanionic therapeutic may achieve effective gene therapy in a subject with reduced or minimal off-target organ toxicity (e.g., reduced or negligible liver toxicity).

### Lipoplex Formulations

Polyanionic therapeutic compounds (e.g., one or more nucleic acids or RNAi agents) may be combined with one or more lipid molecules (e.g., cationic, anionic, or neutral lipids) to produce a lipoplex formulation of this disclosure. The formulation can also include one or more components (e.g., cholesterol, cholesterol-iRGD conjugates, cationic amphiphiles, etc.). Methods of formulating lipids to incorporate nucleic acid therapeutics have been described (see, for example, Betker, J. L. and T. J. Anchordoquy, Relating toxicity to transfection: using sphingosine to maintain prolonged expression in vitro. Mol Pharm, 2015. 12(1): p. 264-73; Betker J L, Anchordoquy T J. Effect of charge ratio on lipoplex-mediated gene delivery and liver toxicity. Therapeutic Delivery 6 (11): 1243-1253 (2015); Watcharanurak, K., et al., Controlling the kinetics of interferon transgene expression for improved gene therapy. J Drug Target, 2012. 20(9): p. 764-9; which are hereby incorporated by reference).

These lipoplexes may include any useful combination of lipid molecules (e.g., a cationic lipid (optionally including one or more cationic lipids, e.g., one or more cationic lipids of this disclosure as described herein and/or optionally including one or more cationic lipids known in the art), a neutral lipid, an anionic lipid, including polypeptide-lipid conjugates and other components that aid in the formation or stability of a lipoplex, as described herein. A person of skill in that art will know how to optimize the combination that favor encapsulation of a particular agent, stability of the lipid formulation, scaled-up reaction conditions, or any other pertinent factor. The formulations of this disclosure may include other components that aid in formation or stability.

The percentage of each component in the formulation can be balanced to produce a lipid component capable of incorporating an RNAi agent and transfecting the agent into a cell. An exemplary lipoplex formulation includes from about 20 mol % to about 60 mol % of one or more C₁₈₋₃₀ saturated fatty acid, 5 mol % to about 30 mol % of one or more cationic amphiphile, 10 mol % to about 20 mol % of one or more sterol, 0.1 mol % to about 5 mol % of one or more sterol-conjugated ligand, and at least one polyanionic therapeutic.

The lipoplexes also include other components that aid in the formation or stability of lipoplexes. Examples of optional additional components include antioxidants (e.g., α-tocopherol or 3-hydroxytoluidine), surfactants, and salts.

Within the scope of this disclosure, charged lipids and nucleic acid components are used to prepare the lipoplex formulations. The corresponding lipoplexes thus carry positive charges on their surface, whereas the nucleic acids are negatively charged by virtue of their phosphate skeleton. Thus, a charge ratio of positively-charged lipids to negatively-charged nucleic acid is formed. The lipids-nucleic acid charge ratio (+/−) indicates the ratio of positive charge of the cationic lipid used to the negative charges of the nucleic acid. It is assumed that all monovalent cationic lipids have one (1) positive charge. This means that the number of moles of cationic lipid put in corresponds to the number of moles of positive charges (this applies to a lipid which has only one (1) positive charge; for polyvalent cationic lipids this has to be taken into consideration in the calculation). The charge carriers of the negative charge on the nucleic acid are the phosphate groups (one negative charge per phosphate group). The lipoplexes of this disclosure may be formulated to have a charge ratio between 0.5 and 4.0. These lipoplexes may also be formulated to have a charge ratio between 0.5 and 1.0.

Formulations with RNAi Agents

The lipoplex formulations of this disclosure are formulated with an RNAi agent by any of the methods described below. The lipoplexes may include an RNAi agent and a lipid molecule and/or one or more components in any useful ratio.

The pharmaceutical compositions of this disclosure can include an RNAi agent in a dose ranging from about 1 mg/kg to about 10 mg/kg of any RNAi agent described here. Exemplary doses include 1 mg/kg, 2 mg/kg, 3 mg/kg, 4 mg/kg, mg/kg, 6 mg/kg, 7 mg/kg, 8 mg/kg, 9 mg/kg, and 10 mg/kg of an RNAi agent in a pharmaceutical composition including these lipoplexes.

### Methods of Preparing Lipoplexes

The lipoplexes of this disclosure can be prepared with any useful process. In one procedure, the components of the formulation (e.g., one or more RNA-binding agents, transfection lipids, or any lipid described herein) are dissolved/dispersed in a solvent (e.g., an aqueous solvent, a non-aqueous solvent, or solvent mixtures thereof). The resultant lipid suspension can be optionally filtered, mixed (e.g., batch mixed, in-line mixed, and/or vortexed), evaporated (e.g., using a nitrogen or argon stream), re-suspended (e.g., in an aqueous solvent, a non-aqueous solvent, or solvent mixtures thereof), freeze-thawed, extruded, and/or sonicated. Furthermore, the lipid suspension can be optionally processed by adding any desired components (e.g., one or more RNAi agents, RNA-binding agents, transfection lipids, and/or any lipids described herein) to produce a final suspension. The one or more desired components can be provided in the same or different solvent as the suspension. For example, the lipid suspension can be provided in a first solvent or solvent system (e.g., one or more aqueous or non-aqueous solvent(s), such as water, water-HCl, water-ethanol, buffer (e.g., phosphate buffered saline (PBS), Hank's balanced salt solution (HBSS), Dulbecco's phosphate-buffered saline (DPBS), Earle's balanced salt solution (EBSS), carbonate, lactate, ascorbate, and citrate, such as 5 mM, 10 mM, 50 mM, 75 mM, 100 mM, or 150 mM)), physiological osmolality solution (290 mOsm/kg, e.g., 0.9% saline, 5% dextrose, and 10% sucrose), saline, methanol, ethanol, n-propanol, isopropanol, n-butanol, isobutanol, tert-butanol, glycerol, ethylene glycol, propylene glycol, polyethylene glycol, chloroform, dichloromethane, hexane, cyclohexane, acetone, ether, diethyl ether, dioxan, isopropyl ether, tetrahydrofuran, or combinations thereof), and the RNAi agent can be provided in a second solvent or solvent system e.g., one or more aqueous or non-aqueous solvent(s), such as water, water-HCl, water-ethanol, buffer (e.g., phosphate buffered saline (PBS), Hank's balanced salt solution (HBSS), Dulbecco's phosphate-buffered saline (DPBS), Earle's balanced salt solution (EBSS), carbonate, lactate, ascorbate, and citrate, such as 5 mM, 10 mM, 50 mM, 75 mM, 100 mM, or 150 mM)), physiological osmolality solution (290 mOsm/kg, e.g., 0.9% saline, 5% dextrose, and 10% sucrose), saline, methanol, ethanol, n-propanol, isopropanol, n-butanol, isobutanol, tert-butanol, glycerol, ethylene glycol, propylene glycol, polyethylene glycol, chloroform, dichloromethane, hexane, cyclohexane, acetone, ether, diethyl ether, dioxan, isopropyl ether, tetrahydrofuran, or combinations thereof). The combination of both components (lipid and nucleic acid) in water allows very weak interactions that would be masked in a buffer and/or saline solution. Once the weak interaction is made between lipid and nucleic acid, the hydrophobic portions of the lipid can hold the complex together when it subsequently encounters an ionic environment (e.g., blood).

Exemplary concentrations of aqueous solvents and/or buffers include from about 4% to about 8% ethanol (e.g., from about 4% to 5%, 5% to 6%, 6%, to 7%, or 7% to 8%), from about 10 mM to about 100 mM citrate (e.g., from about 10 mM to 30 mM, 30 mM to 50 mM, 50 mM to 70 mM, 70 mM to 90 mM, or 90 mM to 100 mM). Any of the solvents or solvent systems can include one or more stabilizers, such as an antioxidant, a salt (e.g., sodium chloride), citric acid, ascorbic acid, glycine, cysteine, ethylenediamine tetraacetic acid (EDTA), mannitol, lactose, trehalose, maltose, glycerol, and/or glucose. In further examples, the one or more RNA-binding agents are introduced into a lipid suspension using a first solvent or solvent system and then followed by addition of one or more transfection lipids in a second solvent or solvent system, where first and second solvents or solvent systems are the same or different (e.g., the first solvent or solvent system is any described herein; and the second solvent or solvent system is any described herein). In particular embodiments, the second solvent or solvent system include one or more aqueous or non-aqueous solvents selected from the group consisting of saline, buffer (e.g., citrate or PBS), water, and ethanol. The final suspension can be optionally separated (e.g., by ultracentrifuge), mixed (e.g., batch mixed, in-line mixed, and/or vortexed), re-suspended, adjusted (e.g., with one or more solvents or buffer systems), sonicated, freeze-thawed, extruded, and/or purified.

### RNAi Agents

RNA interference (RNAi) is a mechanism that inhibits gene expression by causing the degradation of specific RNA molecules or hindering the transcription of specific genes. In nature, RNAi targets are often RNA molecules from viruses and transposons (a form of innate immune response), although it also plays a role in regulating development and genome maintenance. Key to the mechanism of RNAi are small interfering RNA strands (siRNA), which have sufficiently complementary nucleotide sequences to a targeted messenger RNA (mRNA) molecule. The siRNA directs proteins within the RNAi pathway to the targeted mRNA and degrades them, breaking them down into smaller portions that can no longer be translated into protein.

The RNAi pathway is initiated by the enzyme Dicer, which cleaves long, double-stranded RNA (dsRNA) molecules into siRNA molecules, typically about 21 to about 23 nucleotides in length and containing about 19 base pair duplexes. One of the two strands of each fragment, known as the guide strand, is then incorporated into the RNA-induced silencing complex (RISC) and pairs with complementary sequences. RISC mediates cleavage of single-stranded RNA having sequence complementary to the antisense strand of the siRNA duplex. Cleavage of the target RNA takes place in the middle of the region complementary to the antisense strand of the siRNA duplex. The outcome of this recognition event is post-transcriptional gene silencing. This occurs when the guide strand specifically pairs with a mRNA molecule and induces the degradation by Argonaute, the catalytic component of the RISC complex.

The lipoplexes of this disclosure can be used to deliver one or more RNAi agents to a cell in vitro or in vivo (e.g., in a subject). The RNAi agents can include different types of double-stranded molecules that include either RNA:RNA or RNA:DNA strands. These agents can be introduced to cells in a variety of structures, including a duplex (e.g., with or without overhangs on the 3′-terminus), a hairpin loop, or an expression vector that express one or more polynucleotides capable of forming a double-stranded polynucleotide alone or in combination with another polynucleotide. Exemplary RNAi agents include siRNA, shRNA, DsiRNA, and miRNA agents, which are described herein. Generally, these agents are about 10 to about 40 nucleotides in length, and preferred lengths are described below for particular RNAi agents.

Functional gene silencing by an RNAi agent does not necessarily include complete inhibition of the targeted gene product. In some cases, marginal decreases in gene product expression caused by an RNAi agent may translate to significant functional or phenotypic changes in the host cell, tissue, organ, or animal. Therefore, gene silencing is understood to be a functional equivalent and the degree of gene product degradation to achieve silencing may differ between gene targets or host cell type.

siRNA

Small interfering RNA (siRNA) are generally double-stranded RNA molecules of 16 to 30 nucleotides in length (e.g., 18 to 25 nucleotides, e.g., 21 nucleotides) with one or two nucleotide overhangs on the 3′-terminii or without any overhangs. A skilled practitioner may vary this sequence length (e.g., to increase or decrease the overall level of gene silencing). In certain embodiments, the overhangs are UU or dTdT at the 3′-terminus. Generally, siRNA molecules are completely complementary to one strand of a target DNA molecule, since even single base pair mismatches have been shown to reduce silencing. In other embodiments, siRNAs may have a modified backbone composition, such as, 2′-deoxy- or 2′-O-methyl modifications, or any other useful modifications.

shRNA

Short hairpin RNA (shRNA) are single-stranded RNA molecules in which a hairpin loop structure is present, allowing complementary nucleotides within the same strand to form intermolecular bonds. shRNA can exhibit reduced sensitivity to nuclease degradation as compared to siRNA. In certain embodiments, an shRNA have a stem length from 19 to 29 nucleotides in length (e.g., 19 to 21 nucleotides or 25 to 29 nucleotides). In some embodiments, loop size is between 4 to 23 nucleotides in length. shRNA can generally contain one or more mismatches, e.g., G-U mismatches between the two strands of the shRNA stem, without decreasing potency.

DsiRNA

Dicer-substrate RNA (DsiRNA) are double-stranded RNA agents of 25 to 35 nucleotides. Agents of such length are believed to be processed by the Dicer enzyme of the RNA interference (RNAi) pathway, whereas agents shorter than 25 nucleotides generally mimic Dicer products and escape Dicer processing. In some embodiments, DsiRNA has a single-stranded nucleotide overhang at the 3′-terminal of the antisense or sense strand of 1 to 4 nucleotides (e.g., 1 or 2 nucleotides).

Certain modified structures of DsiRNA agents were previously described, as in U.S. Patent Publication No. 2007/0265220, which is incorporated herein by reference. Additional DsiRNA structures and specific compositions suitable for use in the lipoplex formulations of this disclosure are described in U.S. patent application Ser. No. 12/586,283; U.S. Patent Publication Nos. 2005/0244858, 2005/0277610, 2007/0265220, 2011/0021604, 2010/0173974, 2010/0184841, 2010/0249214, 2010/0331389, 2011/0003881, 2011/0059187, 2011/0111056; and PCT Publication Nos. WO 2010/080129, WO 2010/093788, WO 2010/115202, WO 2010/115206, WO 2010/141718, WO 2010/141724, WO 2010/141933, WO 2011/072292, WO 2011/075188, all of which are hereby incorporated by reference. Generally, DsiRNA constructs are synthesized using solid phase oligonucleotide synthesis methods as described for 19-23 mer siRNAs (see U.S. Pat. Nos. 5,804,683; 5,831,071; 5,998,203; 6,117,657; 6,353,098; 6,362,323; 6,437,117; 6,469,158; 6,111,086; 6,008,400; and 6,111,086, which are incorporated herein by reference).

miRNA

MicroRNA (miRNA) are single-stranded RNA molecules of 17 to 25 nucleotides (e.g., 21 to 23 nucleotides) in length. A skilled practitioner may vary this sequence length to increase or decrease the overall level of gene silencing. These agents silence a target gene by binding complementary sequences on target messenger RNA. As used herein, the term “miRNA precursor” is used to encompass, without limitation, primary RNA transcripts, pri-miRNAs and pre-miRNAs. A “miRNA therapeutic” can include pri-miRNA, pre-miRNA, and/or miRNA (or mature miRNA). An siRNA (e.g., a DsiRNA) molecule may present a guide strand that incorporates a miRNA sequence, or is sufficiently homologous to the miRNA sequence to function as said miRNA (rendering such siRNA a “miRNA mimetic”).

Antisense Compounds

Exemplary antisense compounds comprise a consecutive nucleoside length range, wherein the upper end of the range is 50 nucleosides and wherein the lower end of the range is 8 nucleosides. In certain embodiments, the upper end of the range is 35 nucleosides and the lower end of the range is 14 nucleosides. In further embodiments, the upper end of the range is 24 nucleosides and the lower end of the range is 17 nucleosides. In still further embodiments, the antisense compound is 20 consecutive nucleosides. Those skilled in the art will readily recognize that the upper end of the range, as disclosed herein comprises 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 consecutive nucleosides and the lower end of the range comprises 8, 9, 10, 11, 12, 13, 14, 15, 16, 17, 18, 19 or 20 consecutive nucleosides.

Exemplary antisense compounds comprise a stretch of at least 8, optionally at least 12, optionally at least 15 consecutive nucleosides that is sufficiently complementary to a target sequence to interfere with transcription, translation, promote degradation (optionally nuclease-mediated degradation) and/or otherwise disrupt the function (e.g., interfere with the function of an otherwise functional target sequence, e.g., disruption of a promoter, enhancer or other functional nucleic acid target sequence via an antisense compound-mediated mechanism) of the target sequence.

Modifications can be made to antisense compounds and may include conjugate groups attached to one of the termini, selected nucleobase positions, sugar positions or to one of the internucleoside linkages. Possible modifications include, but are not limited to, 2′-fluoro (2′-F), 2′-OMethyl (2′-OMe), 2′-O-(2-methoxyethyl) (2′-MOE) high affinity sugar modifications, inverted abasic caps, deoxynucleobases, and bicyclic nucleobase analogs, such as locked nucleic acids (LNA) and ethylene-bridged nucleic acids (ENA).

### Delivery of a Therapeutic Agent

The formulations of this disclosure may be used to deliver a therapeutic agent (e.g., polyanionic agents, nucleic acids, or RNAi agents) to cells. The agent delivered by the formulation can be used for gene-silencing (e.g., in vitro or in vivo in a subject) or to treat or prophylactically treat a disease (e.g., cancer, such as by immunotherapy) in a subject.

Delivery of a therapeutic agent may be assessed by using any useful method. For example, delivery with a formulation containing the compound of this disclosure may be assessed by 1) knockdown of a target gene or 2) toxicity or tolerability, as compared to a control at an equivalent dose. These assessments can be determined with any useful combination of lipids in the formulation, such as any cationic lipid described herein (e.g., DOTAP, DODMA, DLinDMA, and/or DLin-KC2-DMA) in combination with a compound of this disclosure (e.g., any compound of Formula (I) or in Table 1). In particular embodiments, an improvement of delivery of a therapeutic agent is observed when using a compound of this disclosure, where the improvement is more than 25% (e.g., more than a 2-fold, 5-fold, 10-fold, 100-fold, or 1000-fold improvement in delivery), as compared to a control.

### Delivery of RNAi Agents

RNAi silencing can be used in a wide variety of cells, where HeLa S3, COS7, 293, NIH/3T3, A549, HT-29, CHO-KI and MCF-7 cell lines are susceptible to some level of siRNA silencing. Furthermore, suppression in mammalian cells can occur at the RNA level with specificity for the targeted genes, where a strong correlation between RNA and protein suppression has been observed. Accordingly, a lipoplex of this disclosure, and pharmaceutical formulations thereof, may be used to deliver an RNAi agent to one or more cells (e.g., in vitro or in vivo). Exemplary RNAi agents include siRNA, shRNA, dsRNA, miRNA, and DsiRNA agents, as described herein.

### Cancer Therapy

The lipoplexes of this disclosure can be used to deliver one or more therapeutic agents (e.g., RNAi agents or RNA/DNA-vaccines) to a subject having cancer or at risk of developing a cancer (e.g., an increased risk of at least 10%, 20%, 30%, 40%, 50%, 60%, 70%, 80%, 90%, or 100%). Exemplary cancers include liver cancer (e.g., hepatocellular carcinoma, hepatoblastoma, cholangiocarcinoma, angiosarcoma, or hemangiosarcoma) or neuroblastoma. Exemplary neoplastic diseases and associated complications include, but are not limited to, carcinomas (e.g., lung, breast, pancreatic, colon, hepatocellular, renal, female genital tract, squamous cell, carcinoma in situ), lymphoma (e.g., histiocytic lymphoma, non-Hodgkin's lymphoma), MEN2 syndromes, neurofibromatosis (including Schwann cell neoplasia), myelodysplastic syndrome, leukemia, tumor angiogenesis, cancers of the thyroid, liver, bone, skin, brain, central nervous system, pancreas, lung (e.g., small cell lung cancer, non-small cell lung cancer (NSCLC)), breast, colon, bladder, prostate, gastrointestinal tract, endometrium, fallopian tube, testes and ovary, gastrointestinal stromal tumors (GISTs), prostate tumors, mast cell tumors (including canine mast cell tumors), acute myeloid myelofibrosis, leukemia, acute lymphocytic leukemia, chronic myeloid leukemia, chronic lymphocytic leukemia, multiple myeloma, melanoma, mastocytosis, gliomas, glioblastoma, astrocytoma, neuroblastoma, sarcomas (e.g., sarcomas of neuroectodermal origin or leiomyosarcoma), metastasis of tumors to other tissues, and chemotherapy-induced hypoxia.

### Administration and Dosage

This disclosure also provides pharmaceutical compositions that contain a therapeutically effective amount of a lipoplex formulation, such as a formulation including a therapeutic agent (e.g., an RNAi agent). The composition can be formulated for use in a variety of drug delivery systems. One or more physiologically acceptable excipients or carriers can also be included in the composition for proper formulation. Suitable formulations for use in this disclosure are found in Remington's Pharmaceutical Sciences, Mack Publishing Company, Philadelphia, Pa., 17th ed., 1985. For a brief review of methods for drug delivery, see, e.g., Langer, Science 249:1527-1533, 1990.

These pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in this disclosure, by such means as depot injections or erodible implants or components. Thus, this disclosure provides compositions for parenteral administration that comprise the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. This disclosure also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a patient with a clinically determined predisposition or increased susceptibility to development of a tumor or cancer. A lipoplex of this disclosure can be administered to the patient (e.g., a human) in an amount sufficient to delay, reduce, or preferably prevent the onset of clinical disease or tumorigenesis. In therapeutic applications, compositions are administered to a patient (e.g., a human) already suffering from a cancer in an amount sufficient to cure or at least partially arrest the symptoms of the condition and its complications. An amount adequate to accomplish this purpose is defined as a “therapeutically effective dose,” an amount of a compound sufficient to substantially improve some symptom associated with a disease or a medical condition. For example, in the treatment of cancer, an agent or compound which decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Amounts effective for this use may depend on the severity of the disease or condition and the weight and general state of the patient, but generally range from about 0.5 mg to about 3000 mg of the agent or agents per dose per patient. Suitable regimes for initial administration and booster administrations are typified by an initial administration followed by repeated doses at one or more hourly, daily, weekly, or monthly intervals by a subsequent administration. The total effective amount of an agent present in the compositions of this disclosure can be administered to a mammal as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more agents present within the compositions of this disclosure and used in the methods of this invention applied to mammals (e.g., humans) can be determined by the ordinarily-skilled artisan with consideration of individual differences in age, weight, and the condition of the mammal. The agents of this disclosure are administered to a subject (e.g., a mammal, such as a human) in an effective amount, which is an amount that produces a desirable result in a treated subject (e.g., the slowing or remission of a cancer or neurodegenerative disorder). Such therapeutically effective amounts can be determined empirically by those of skill in the art.

The patient may also receive an agent in the range of about 0.1 to 3,000 mg per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) mg dose per week. A patient may also receive an agent of the composition in the range of 0.1 to 3,000 mg per dose once every two or three weeks.

The amount (dose) of the lipoplex formulation and polyanionic therapeutic (e.g., miRNA) that is to be administered can be determined empirically. In certain embodiments, effective knockdown of gene expression is observed using 0.0001-10 mg/kg animal weight of nucleic acid therapeutic and 0.001-200 mg/kg animal weight delivery formulation. An exemplary amount in mice is 0.1-5 mg/kg nucleic acid therapeutic and 0.7-100 mg/kg delivery formulation. Optionally, about 1-50 mg/kg delivery formulation is administered. The amount of therapeutic (e.g., miRNA) is easily increased because it is typically not toxic in larger doses.

In these embodiments, doses can be administered daily over a period of days, weeks, or longer (e.g., between one and 28 days or more), or only once, or at other intervals, depending upon, e.g., acute versus chronic indications, etc. In exemplary embodiments, doses can be administered every 24 to 72 hours (i.e., every day or every 2 or 3 days).

Single or multiple administrations of the compositions of this disclosure comprising an effective amount can be carried out with dose levels and pattern being selected by the treating physician. The dose and administration schedule can be determined and adjusted based on the severity of the disease or condition in the patient, which may be monitored throughout the course of treatment according to the methods commonly practiced by clinicians or those described herein.

### Kits

In another aspect, this disclosure provides kits that include components of a lipid complex that may be used to form a lipoplex formulation of this disclosure, such as at least one naturally-occurring cationic amphiphile, at least one C₁₈₋₃₀ saturated fatty acid, and cholesterol. The kits may include permutations of these lipid complex components and such permutations are expressly within the scope of this disclosure.

The kits may also include at least one polyanionic therapeutic (e.g., a nucleic acid such as miRNA or siRNA). But one specific kit embodiment contemplated in this disclosure is a kit containing any one or more of the kit components listed above or below, but absent a specific polyanionic therapeutic, i.e., this includes a kit intended for preparing a lipoplex of this disclosure in which the polyanionic therapeutic is supplied by the skilled artisan that obtains the kit in order to prepare effective lipoplexes of this disclosure using their own RNAi molecule/strategy.

The kits may also include one or more other elements including, but not limited to, instructions for use; other reagents, e.g., a diluent, devices or other materials for preparing the lipoplexes and/or pharmaceutical compositions for administration; pharmaceutically acceptable carriers; and devices or other materials for administration to a subject. Instructions for use can include instructions for therapeutic application, including suggested dosages and/or modes of administration, e.g., in a human subject, as described herein.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

### EXAMPLES

### Example 1

Gene delivery offers the potential to cure diseases that are currently very difficult to treat with conventional pharmaceuticals. Although many different delivery vehicles continue to be investigated, cationic lipid-based vectors (i.e., lipoplexes) have shown significant promise and are the most widely studied. It is generally recognized that the physical properties of the delivery system (e.g., size, charge) can have a significant effect on delivery both in cell culture and in vivo. To maximize delivery, the physical properties of nucleic acid-containing delivery vehicles are commonly varied during preparation by altering the relative amounts of cationic agent to nucleic acid, i.e., the +/− charge ratio. In addition, the adsorption of serum proteins can dramatically modify the physical properties of the delivery system such that the resulting protein-nanoparticle complex which ultimately encounters the target cell differs substantially from the original in vitro preparation. Nonetheless, the charge ratio is one of the first parameters that is optimized in initial experiments, and positive charge ratios 2) generally provide superior transfection. However, it is important to recognize that the cationic component is responsible for toxicity, and thus formulations possessing higher charge ratios typically exhibit greater toxicity. Furthermore, our recent study has demonstrated that the toxicities measured after a 24-48 h transfection experiment can be misleading, and even a brief exposure to cationic delivery vehicles can initiate a prolonged reaction that does not result in death (as measured by conventional assays) for more than a week. Therefore, it may be beneficial to formulate delivery vehicles with lower levels of cationic agent (i.e., at lower charge ratios), such that toxicity is reduced.

Our previous work has documented the effect of charge ratio on the protein corona that is recruited from serum. While an effect of charge ratio on the protein corona is not surprising, we did not observe any adsorbed proteins that were specifically responsible for changes in transfection rates. But it is commonly observed that preparation at lower charge ratios results in reduced transfection rates. In fact, studies have shown that formulations possessing high charge ratios contain free (uncomplexed) cationic agents that contribute to transfection, presumably by permeabilizing cells in culture. Not surprisingly, these free cationic components that facilitate gene delivery in vitro contribute significantly to toxicity.

It is important to point out that in vitro transfection experiments virtually always employ a constant amount of nucleic acid complexed to increasing amounts of cationic agent, i.e., to achieve different charge ratios. Considering that toxicity of the cationic agent is likely to be the limiting factor governing dosing of a gene delivery vehicle, it could be argued that experiments should be designed to assess the extent to which transfection can be achieved at a constant level of cationic agent. In so doing, investigators could determine a tolerable level of cationic agent in a manner analogous to a Phase I clinical trial, and then maximize the amount of DNA delivered with that dose of cationic agent. In addition, the preparation of gene delivery vehicles involves a spontaneous electrostatic association of negatively-charged nucleic acid with cationic reagents. Considering this method of production, it should not be surprising that the properties of the particles produced in this process are highly dependent on mixing conditions, including the concentration of cationic components. It is well established that more concentrated mixing conditions produce particles with larger diameters, which are generally considered less desirable. Thus, preparation of gene delivery vehicles at lower charge ratios not only reduces toxicity, but also allows smaller-sized particles to be produced.

The current study investigates in vitro transfection of a murine mammary carcinoma cell line (4T1) with lipoplexes prepared at different charge ratios. It is important to point out that lipoplexes are incubated in full-strength serum (a 1:1 v/v mixture to achieve a final serum concentration of 50%) to simulate interactions in the blood upon intravenous injection prior to conducting transfection assays. As suggested above, separate experiments are performed at both constant nucleic acid levels and at constant cationic lipid. By designing experiments in this way, we were able to identify an optimal anionic lipoplex (i.e., charge ratio=0.5) as well as an optimized cationic lipoplex (i.e., charge ratio=4). Five formulations prepared at these charge ratios were then compared in vivo to assess circulation half-life, tumor accumulation, reporter gene expression, biodistribution, and liver toxicity.

Materials and Methods

Cholesterol, N-(1-(2, 3-dioleoyloxy) propyl)-N, N, N-trimethylammonium chloride (DOTAP), diarachidoyl-sn-glycero-3-phosphocholine (DAPC), egg phosphatidylcholine and sphingosine were purchased from Avanti Polar Lipids (Alabaster, Ala.) and used to prepare liposomes. Lipofectamine 2000 was purchased from Invitrogen (Carlsbad, Calif.) and used according to the manufacturer's instructions. Lipoplexes were then prepared at different+/− charge ratios by mixing equal volumes of a modified pSelect-LucSh (Invivogen, San Diego, Calif.) plasmid encoding luciferase with the suspended liposomes as previously described.

### Transfection Protocol

Murine mammary carcinoma cells (4T1; ATCC #CRL-2539) were cultured at 37° C., 5% carbon dioxide with 100% humidity in Minimum Essential Media (MEM), 10% fetal bovine serum (FBS), 50 U/ml penicillin, 50 μg/ml streptomycin (all media from Cellgro MediaTech Inc., a Corning Acquisition, Manassas, Va.) as previously described. For in vitro experiments, lipoplexes were pre-incubated 1:1 v/v in FBS (i.e., 50% FBS to mimic in vivo serum protein conditions) for 30 minutes prior to dilution to 10% FBS with 100% MEM, and then administered to cells for transfection. Formulations were applied to the center of each well and allowed to incubate for 4 hours. After 4 hours, the transfection media was carefully removed, cells were washed with PBS, and then returned to 10% FBS growth media. After 48 hours, cells were lysed with 30 μl Promega lysis buffer in the −80° C. freezer according to manufacturer's instructions (Promega, Madison, Wis.). The lysates were also assayed for protein content with a Bio-Rad protein assay (Bio-Rad, Hercules, Calif.) on a 96-well THERMOmax plate reader (Molecular Devices, Sunnyvale, Calif.). Luminescence was quantified using a Monolight Luminometer according to manufacturer's instructions (BD Biosciences, San Jose, Calif.).

### Animal Studies

Lipoplexes prepared at +/−=0.5 or +/−=4 were diluted 1:1 (v/v) with 12% hydroxyl ethyl starch (MW 250,000, Fresenius; Linz, Austria) prior to administration, and 50 μg DNA was injected via tail vein as previously described. Prior to treatment with lipoplexes, female immunocompetent Balb/c mice 4-8 weeks old were acquired from Jackson Labs (Bar Harbor, Me.) and inoculated in each shoulder with 1×10⁶ 4 T1 murine mammary carcinoma cells. Luciferase expression was monitored in extracted tissues with Promega Luciferase Assay Reagents (Madison, Wis.) as previously described. To determine delivery of plasmid DNA to organs, tissues were harvested 24 h after lipoplex administration, and flash frozen in liquid nitrogen. Tissues were then thawed, weighed, and DNA extracted using the Qiagen DNeasy Blood and Tissue Kit (Qiagen, Germantown, Md.). qPCR was also performed on the samples using QuantiTech RTPCR Kit (Qiagen, Germantown, Md.) on a Applied Biosystems 7500 RTPCR instrument (Grand Island, N.Y.) as previously described. A standard curve of pure plasmid was used for quantification in addition to amplicon efficiency factors that account for amplification that is not perfectly efficient. Plasmid levels and luciferase expression were compared by using a one-way ANOVA, and p<0.05 was considered significant.

### Blood Clearance

To determine blood clearance of the lipoplexes, individuals were bled at 5, 30, 60, and 240 minutes using their submandibular veins as previously described. Blood was collected and spun (2,000×g for 10 minutes) to remove red blood cells. Each sample was then prepped for qPCR using Qiagen DNeasy Blood and Tissue Kit. qPCR was performed using a standard curve of pure plasmid as previously described. The resulting curves were fit using a first order logarithmic function within the Excel program with R² ranging from 0.76-0.94.

### Liver Toxicity

Liver toxicity was assessed by monitoring the levels of alanine aminotransferase (ALT) 24 h after lipoplex administration. Blood was collected from the submandibular vein of experimental animals as described above, and enzyme levels were assessed with an Alanine Transaminase Activity Assay from Abcam PLC (Cambridge, Mass.) according to the manufacturer's instructions.

Results

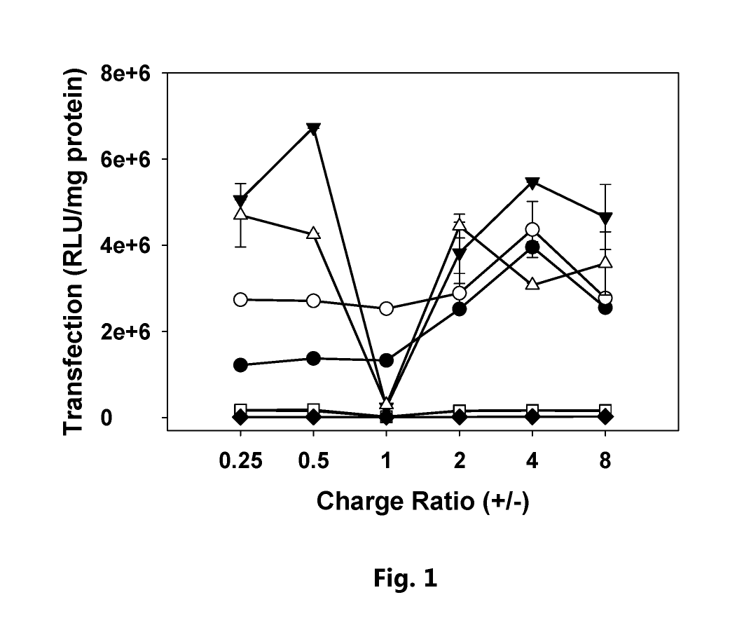

Our initial transfection experiments were conducted with murine mammary carcinoma cells (4T1) in culture. As is typical for in vitro optimization experiments, different charge ratios were prepared by varying the amount of lipid complexed with a constant amount of DNA. In addition, we compared transfection rates achieved with six different cholesterol-containing lipoplex formulations to that of lipofectamine. As noted above, lipoplex formulations were exposed to full-strength serum to simulate conditions upon intravenous administration prior to performing transfection experiments. It is the exposure to serum that greatly reduces the ability of lipofectamine to transfect cells, consistent with our previous studies (FIG. 1). Relatively low transfection rates were also observed for lipoplexes formulated with equimolar amounts of cholesterol and cationic lipid (i.e., DOTAP or sphingosine), regardless of charge ratio. In contrast, transfection with the other four formulations showed a strong dependence on charge ratio. When considering these cationic lipoplexes (i.e., charge ratio >1), three of the four lipoplex formulations exhibited a progressive increase as more cationic agent was included, with a peak transfection rate at charge ratio 4 (FIG. 1). The lipoplex formulation incorporating DAPC and sphingosine showed a maximum transfection efficiency at charge ratio=2, with slightly lower levels at higher charge ratios. Surprisingly, anionic lipoplexes (i.e., charge ratio <1) were also able to efficiently transfect cells, and DAPC-containing formulations displayed high transfection rates at charge ratios 0.5 and 0.25 (FIG. 1). The two formulations prepared at high cholesterol content (80 mole %) did not exhibit this effect, and transfection was essentially constant at anionic charge ratios.

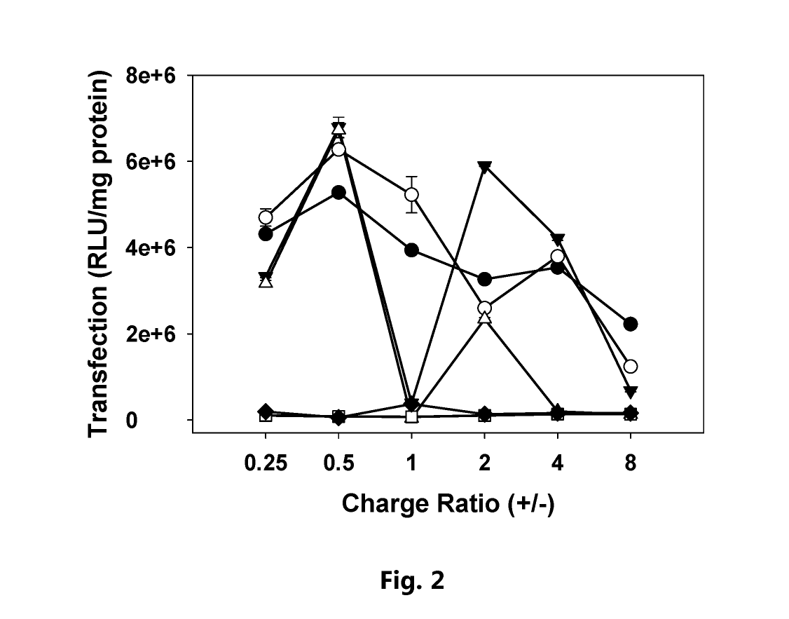

Because the toxicity of the cationic components can limit transfection efficiency and may ultimately limit dosing, a separate in vitro experiment was performed in which the cationic lipid was held constant, and different charge ratios were achieved by altering the amount of DNA incorporated into the preparation (FIG. 2). Under these conditions, a distinct maximum transfection efficiency is observed at charge ratio 0.5 with the four promising formulations from FIG. 1. These lipoplexes showed somewhat different peak transfections as cationic formulations, but three of the four showed good transfection at charge ratio 4, with reduced rates observed at the higher charge ratio (+/−=8; compare FIGS. 1 and 2). Because of the peak transfections observed at charge ratios 0.5 and 4, lipoplexes formulated at these charge ratios were further investigated in vivo.

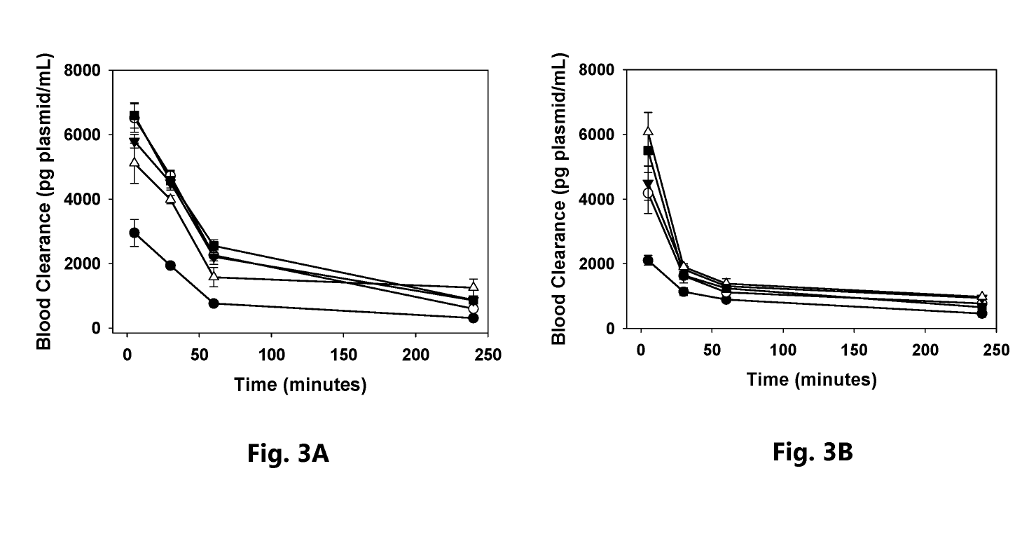

As detailed in the methods, the levels of plasmid in the blood were determined at different time points for each of the four promising formulations and also lipofectamine at constant doses of DNA (FIGS. 3A and 3B). All formulations were cleared rapidly, and computed half-lives were <2.5 h (Table 1).

TABLE 1
Circulation Half-Lives (minutes).

| Formulation | Charge Ratio = 4 | Charge Ratio = 0.5 |
|---|---|---|
| Lipofectamine | 48.6 | 70.1 |
| 4 Cholesterol:1 DOTAP | 57.2 | 36.9 |
| 2 Cholesterol:3 DOTAP:5 | 68.2 | 43.5 |
| DAPC | | |
| 4 Cholesterol:1 Sphingosine | 66.9 | 30.3 |
| 2 Cholesterol:3 Sphingosine:5 | 59.7 | 48.9 |
| DAPC | | |

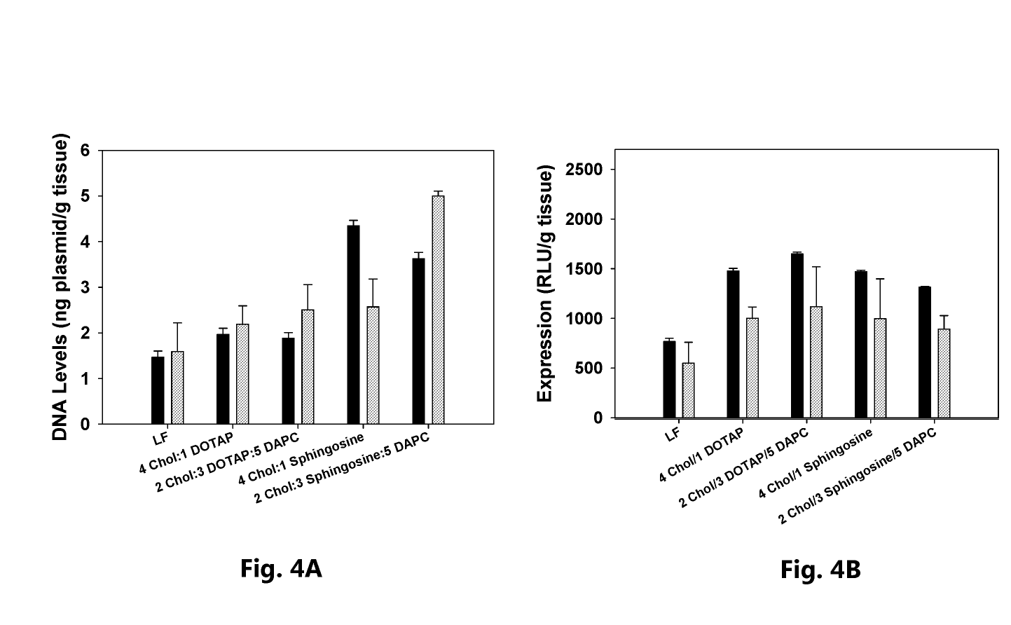

As might be anticipated considering the enhanced plasmid protection expected by incorporating greater amounts of lipid, circulation half-lives were consistently longer at charge ratio 4 as compared to charge ratio 0.5, with the exception of the lipofectamine formulation (Table 1). In spite of the longer half-lives observed at the higher charge ratios, delivery of plasmid to the tumor was comparable for all formulations, with somewhat higher delivery observed with anionic lipoplexes formulated with sphingosine and DAPC (FIG. 4A). Conversely, reporter gene expression (i.e., luciferase) in the tumor was generally higher with the cationic complexes, but still comparable between charge ratios (FIG. 4B).

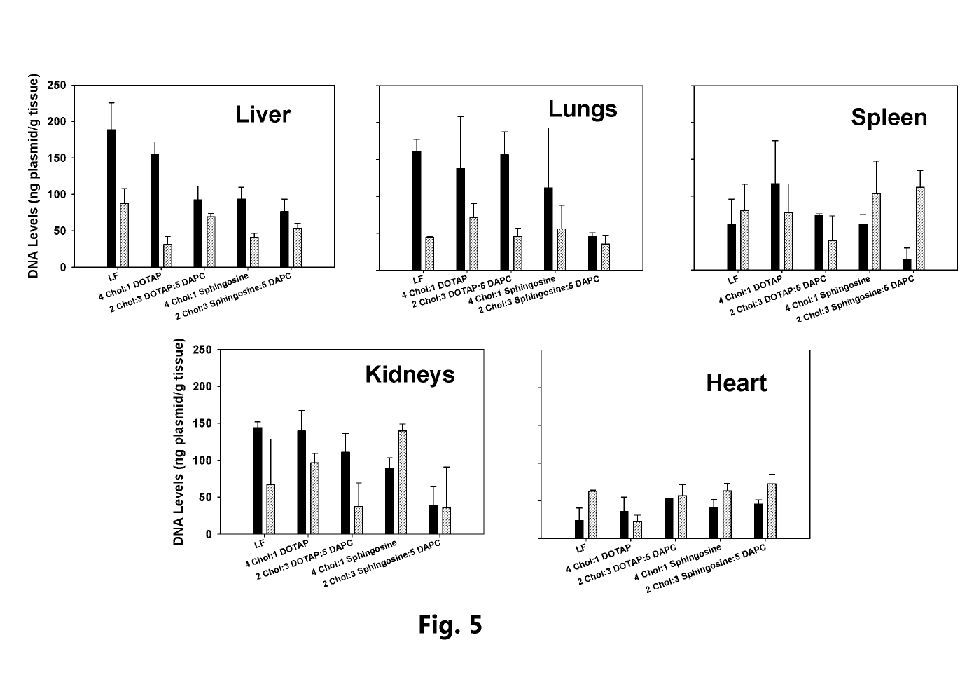

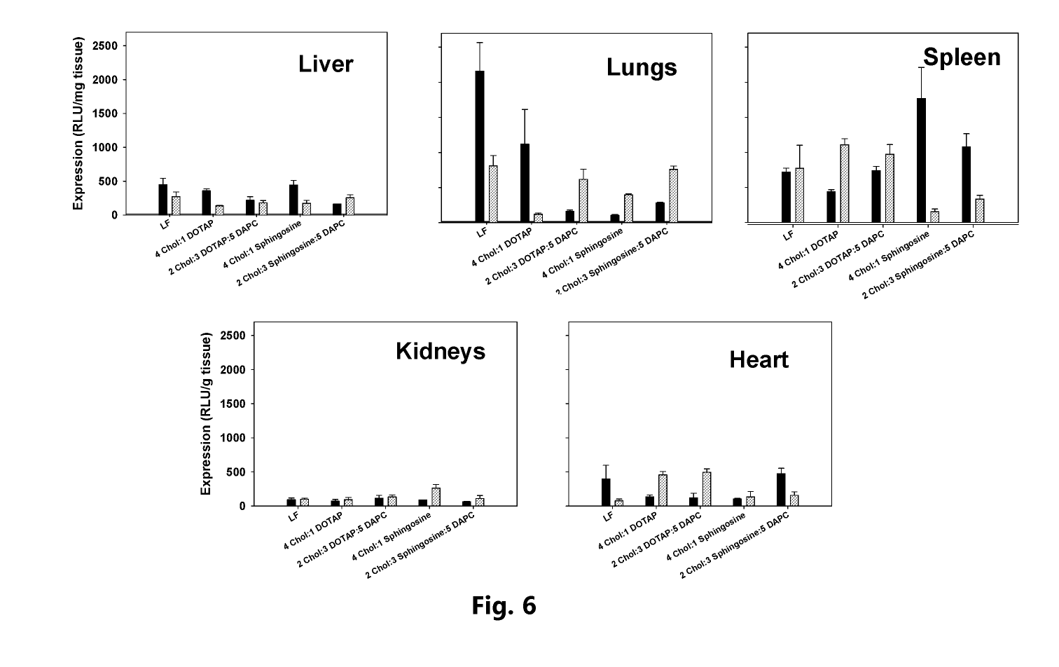

Only small differences among formulations were observed when delivery to other organs was evaluated (FIG. 5). However, cationic lipoplexes generally displayed greater accumulation in the liver and lungs as compared to anionic lipoplexes. Overall, plasmid levels in the spleen, kidney and heart were comparable between the two charge ratios. Similar to that seen in the tumor, expression levels did not correlate with differences in plasmid levels, and expression in the spleen was generally higher than that seen in other organs (FIG. 6). Notably, the highest expression observed was exhibited by the cationic lipofectamine formulation in the lung.

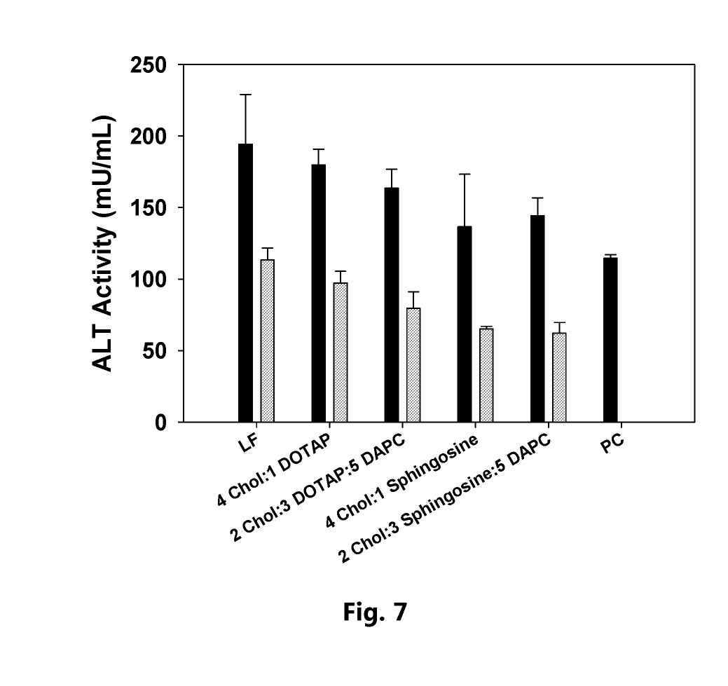

To evaluate the liver toxicity of the formulations, assays were performed to quantify the activity of a liver enzyme (ALT) that is a commonly-used marker for liver damage (FIG. 7). The results indicate a marked increase in ALT activity at charge ratio 4, consistent with the higher levels of liver accumulation noted for the cationic lipoplexes (FIG. 5). We found it somewhat surprising that very little difference in liver toxicity (as reflected by ALT activity) was observed among formulations prepared with a multi-valent cationic lipid (lipofectamine), a mono-valent cationic lipid (DOTAP), and a naturally-occurring, partially-charged cationic amphiphile (sphingosine). These results would seem to suggest that acute liver toxicity is primarily determined by the amount of lipid material that gets deposited in the liver, rather than the chemical nature of the lipids in the nanoparticle. This suggestion that liver toxicity is not attributable to specific lipid species is consistent with experiments showing that the same dose of egg phosphatidylcholine liposomes elicit a comparable elevation in ALT (FIG. 7).

### DISCUSSION

The effect of charge ratio on transfection is well established, but the majority of studies utilize cationic lipoplexes because the positive charge is thought to facilitate interactions with the negatively-charged cell surface. However, it is important to note that serum proteins readily adsorb to cationic particles and reverse their charge. Therefore, it is difficult to understand how an electrostatic interaction with the target cell surface could play a significant role after intravenous administration. In this context, higher charge ratios likely facilitate in vivo gene delivery by providing stronger interactions with the nucleic acid that result in better protection against nucleases, and enhance resistance to dissociation in the blood. The longer half-lives exhibited by lipoplexes formulated with higher amounts of lipid (i.e., +/−=4) are consistent with this suggestion. In our in vitro studies, lipoplexes were incubated in full-strength serum prior to cell exposure, and thus the potential for electrostatic interactions with the cell surface to facilitate in vitro transfection is greatly diminished. In spite of this, we still observe a dramatic effect of charge ratio, and peak transfection efficiencies are observed with both cationic (+/−=4) and anionic (+/−=0.5) lipoplexes (FIGS. 1 and 2). Consistent with our previous studies, lipoplexes possessing a cholesterol domain exhibited much higher transfection rates than those formulated at equimolar amounts of cholesterol and cationic lipid. Thus, only domain-containing lipoplexes were selected for further investigation in an animal model.

When these formulations were investigated in vivo, the greater amount of lipid associated with a higher charge ratio was likely responsible for the longer circulation half-lives as compared to anionic lipoplexes (FIG. 3; Table 1). Despite the increased circulation half-life observed with cationic lipoplexes (+/−=4), tumor accumulation and expression were generally comparable among formulations (FIGS. 4A and 4B). This result is inconsistent with the widely-held notion that longer circulation times result in greater tumor accumulation via the enhanced permeation and retention effect as described for macromolecules. However, the conventional understanding of this effect has recently been questioned, and studies have shown that association with the tumor vasculature can occur much more rapidly than penetration into the tumor mass. Furthermore, the rate of penetration is progressively slowed as particle size increases, suggesting that circulation lifetime may have more significant effects on tumor accumulation with very small delivery systems, i.e., when blood half-life is comparable to the rate of tumor penetration. In addition, our recent studies utilizing the same tumor and lipoplex in different mouse models where we observe comparable tumor uptake despite a 3-fold difference in circulation half-life are consistent with this hypothesis.

The data in FIGS. 3A, 3B and 5 also show that the cationic complexes with longer half-lives also exhibit greater accumulation in the liver and lungs, with comparable deposition in the spleen (as compared to anionic complexes). This trend was evident across formulations, and suggests that particle uptake by the liver, lungs and spleen do not strictly determine circulation half-life of the lipoplexes investigated in this study. These findings are inconsistent with the idea that circulation half-life is solely determined by clearance in the RES organs. Admittedly, the circulation lifetimes are brief (t_{1/2}<2.5 h) when compared to the time at which organ deposition was measured (24 h), but these observations challenge conventional notions about clearance and warrant further investigation.

It is important to note that the plasmid levels observed in tumors were generally 50-100 fold lower than that observed in other organs (per gram tissue). The fact that our lipoplexes did not contain a targeting ligand likely contributes to the observed low accumulation in the tumor. Despite the relatively low levels of plasmid, overall expression in the tumor was higher than that seen in the other tissues (compare FIGS. 4B and 6). This effect might be expected if a tumor-specific promoter was employed, but our plasmid was designed for prolonged expression that should be independent of cell origin. Therefore, while the low plasmid levels in the tumor can likely be enhanced by employing a targeting ligand, the mechanism(s) responsible for the relatively high tumor expression is unknown.

In conclusion, our results in immunocompetent tumor-bearing Balb/c mice indicate that formulation of domain-containing lipoplexes at anionic charge ratios (+/−=0.5) provides comparable gene delivery to the tumor, and decreased liver and lung accumulation as compared to cationic charge ratios. The decreased liver accumulation correlated with a reduced liver toxicity observed with all formulations at the lower charge ratio. This might be expected considering that the lipid dose was 8-fold lower with the +/−=0.5 formulation as opposed to the +/−=4 formulation. However, we also observed no effect of lipid species on ALT levels; the multi-valent lipofectamine formulation exhibited comparable toxicity to that of the mono-valent lipids. This result was especially surprising considering that some formulations utilized a naturally-occurring cationic amphiphile (sphingosine) that is minimally charged under physiological conditions, but is consistent with the fact that the same dose of liposomes composed solely of phosphatidylcholine elicited a similar response. In addition, the decreased liver and lung accumulation observed at the lower charge ratio did not result in enhanced circulation lifetimes. On the contrary, lipoplexes formulated at the lower charge ratio exhibited more rapid clearance as compared to cationic formulations. Surprisingly, this reduced circulation half-life did not result in decreased tumor accumulation at 24 h, consistent with our previous work. Future studies will investigate the incorporation of targeting ligands as well as multiple administrations to enhance therapeutic gene expression in the tumor.

These data demonstrate that formulation of these lipoplexes at lower levels of cationic agent (i.e., low+/− charge ratios) results in lipoplexes with superior transfection activity in vitro and efficient delivery in vivo; that anionic lipoplexes exhibit reductions in circulation times, liver toxicity, liver and lung accumulation compared to cationic lipoplexes; and that, despite reduced circulation times, delivery to the tumor is not reduced by utilizing lipoplexes formulated at anionic charge ratios (+/−=0.5).

### Example 2

Repetitive administration is routinely used to maintain therapeutic drug levels, but previous studies have documented an accelerated blood clearance of some lipid-based delivery systems under these conditions. To assess the effect of repetitive administration, non-PEGylated lipoplexes (+/−=0.5) were administered four times via tail vein injection at 3-day intervals to immunocompetent Balb/c mice bearing 4T1 tumors. This study measured the effect of repeat administration of non-targeted lipoplexes on clearance, plasmid distribution, reporter gene expression, and liver toxicity.

Nonviral gene delivery systems suffer from inefficient delivery compared to their viral counterparts. But synthetic delivery systems have the potential for repeat administration due to the lack of a specific immune response to the vector, and this strategy allows delivery from nonviral systems to be greatly enhanced. Furthermore, it is generally recognized that both the nucleic acid component as well as the nonviral delivery system can be immunostimulatory, and this can affect delivery after successive administrations. Therefore, we have taken great lengths to diminish the adverse response to our delivery system by utilizing minimal amounts of naturally-occurring lipids, reducing the CpG content of the plasmid, and avoiding the use of PEGylation. This study investigated the effects of repeat administration of a lipoplex on clearance, organ accumulation, and liver toxicity. We observed effects on each of these parameters that are not additive and are thereby inconsistent with a conventional pharmacokinetic profile. These results demonstrate that the correlation between clearance, tissue accumulation, reporter gene expression, and liver toxicity are not straightforward, but are surprisingly beneficial in a delivery system. In addition, our in vivo imaging demonstrates that reporter gene expression is widely distributed throughout the mouse 24 h after each tail vein injection, but predominantly confined to the tumor at later times (72 h).

### Materials and Methods

Lipoplex Preparation and Luciferase Expression

Sphingosine, cholesterol, and 1,2-diarachidoyl-sn-glycero-3-phosphocholine (DAPC) were purchased from Avanti Polar Lipids (Alabaster, Ala.) and used to prepare liposomes at a 3:2:5 mole ratio (respectively) as previously described (Betker J L, Anchordoquy T. J. Relating toxicity to transfection: using sphingosine to maintain prolonged expression in vitro. Molecular pharmaceutics. 2015; 12:264-73). Liposomes were then mixed with a modified (CMV removed, ROSA26 added) pSelect-LucSh (Invivogen, San Diego, Calif.) plasmid encoding luciferase at a charge ratio of 0.5. These modifications to the plasmid have been shown to prolong expression for weeks to months. The resulting lipoplexes have a diameter of 280.9±10.8 nm and were diluted 1:1 (v/v) with 12% hydroxyl ethyl starch (MW 250,000, Fresenius; Linz, Austria) prior to administration. The use of hydroxyl ethyl starch at a final concentration of 6% (w/v) serves to adjust the tonicity and results in more consistent, but not increased, delivery. Fifty micrograms of DNA was injected via tail vein as previously described. Each mouse received a series of four injections at three-day intervals. Prior to treatment with lipoplexes, female immunocompetent Balb/c 6-10 weeks old were acquired from Jackson labs (Bar Harbor, Me.) and inoculated in each shoulder with one million 4T1 murine mammary carcinoma cells (ATCC #CRL-2539). Luciferase expression was monitored in extracted tissues with Promega Luciferase Assay Reagents (Madison, Wis.).

### Determination of Plasmid Levels in Tissues

To determine delivery of plasmid DNA to mouse tissues, animals were sacrificed 24 h after the first and fourth intravenous administration of lipoplexes, and their organs were harvested and flash frozen in liquid nitrogen. Organs were then thawed, weighed, and DNA was extracted using the Qiagen DNeasy Blood and Tissue Kit (Qiagen, Germantown, Md.). Quantitative PCR (qPCR) was then performed on the samples using QuantiTech RTPCR Kit (Qiagen, Germantown, Md.) on an Applied Biosystems 7500 RTPCR instrument (Grand Island, N.Y.). A standard curve of pure plasmid was used for quantification as well as amplicon efficiency factors that account for amplification that is not perfectly efficient (as suggested by the Applied Biosystems 7500 Manual referencing Fenster et al. “Real-Time PCR.” Current Protocols Essential Laboratory Techniques, 2009: 10.3.1-10.3.33).

### Clearance

To determine blood clearance of the lipoplexes, individual mice were bled at 5, 30, 60, 240, and 1440 minutes after each injection using their submandibular veins. Briefly, mice were anesthetized using isoflurane and then bled by lancing the submandibular vein on the cheek per standard protocol. Blood was collected in tubes containing sodium citrate (anticoagulant) and spun (2,000×g for 10 minutes) to remove red blood cells, and the resulting plasma was then prepped for qPCR using Qiagen DNeasy Blood and Tissue Kit. qPCR was performed as previously described using the QuantiTech RTPCR Kit (both Qiagen, Germantown, Md.) and a standard curve of pure plasmid.

### Extraction Efficiency

In order to determine the extraction efficiency of the Qiagen DNeasy Blood and Tissue Kit (Qiagen, Germantown, Md.), plasmid DNA (50 μg) was injected into organs freshly harvested from Balb/c mice. Each organ was processed per the Qiagen DNeasy protocol, and plasmid quantified by qPCR. A standard curve of pure plasmid was used, and the calculated extraction efficiencies were used to adjust DNA recoveries in our experiments.

### In Vivo Imaging

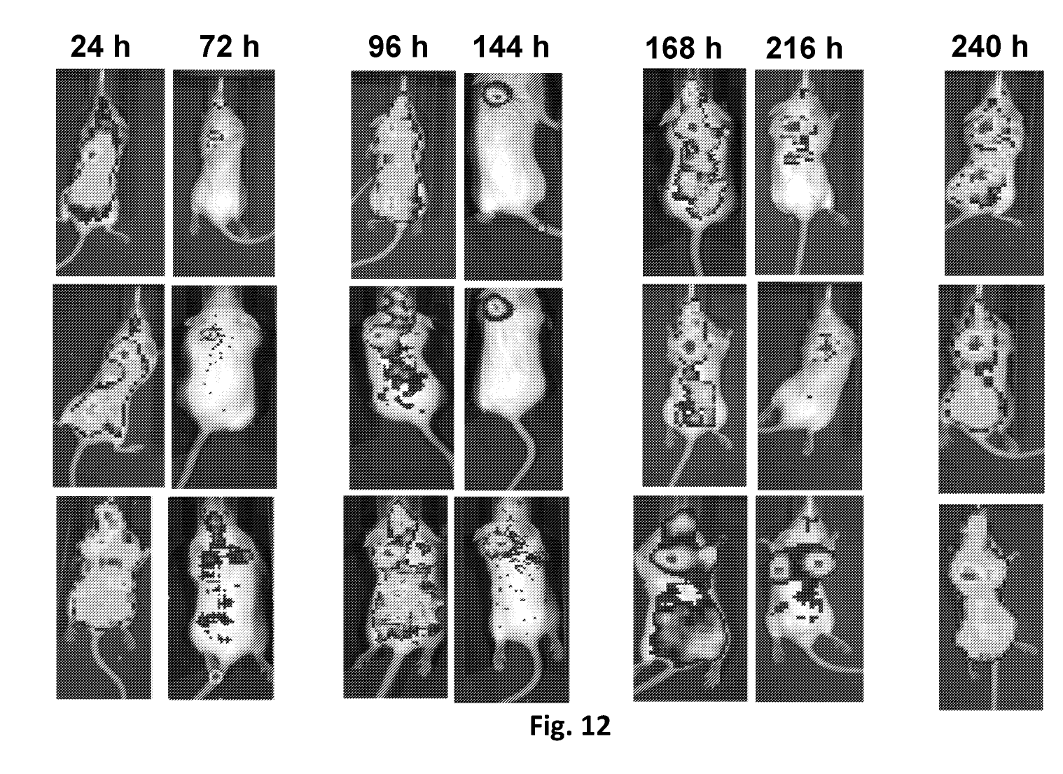

Luciferase expression in Balb/c mice was imaged at different times (see FIG. 12) using the IVIS imaging system (Xenogen Corp., Alameda, Calif.). Briefly, tumor-bearing mice were injected intraperitoneally with D-firefly luciferin substrate (150 mg/kg; Xenogen Corp.) ten minutes prior to anesthetizing mice (2.5% isoflurane in 5 L O2/min). At each timepoint, anesthetized mice were placed in a light-tight chamber and imaging is performed. Images were processed with Living Image software, and representative images were selected for the panels in FIG. 12. After the final timepoint (240 h, four injections), tumor volumes in mice were 200-500 mm3, and tissues were extracted as described above.

### Liver Toxicity

A separate set of mice subjected to the same treatment protocol were used for ALT measurements, and liver toxicity was assessed by monitoring the levels of alanine aminotransferase (ALT) after the first, second, and fourth injections of either lipoplexes or saline. Blood was collected from the submandibular vein of experimental animals as described above, and enzyme levels were assessed with an Alanine Transaminase Activity Assay from Abcam PLC (Cambridge, Mass.).

### Results

Clearance

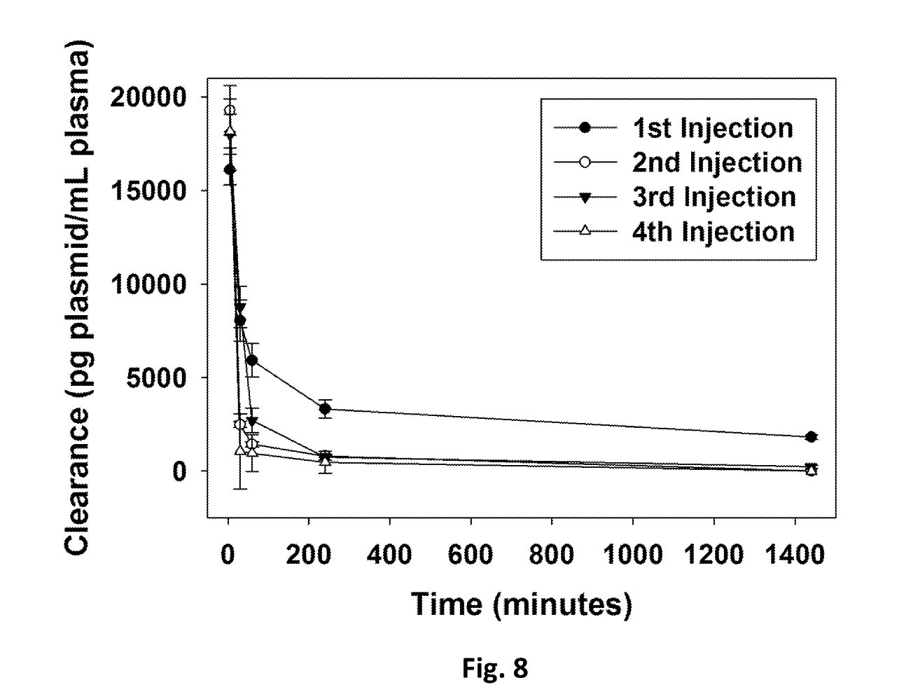

Considering the accelerated blood clearance phenomenon in which subsequent doses of PEGylated liposomes are cleared more rapidly than the initial dose, we assessed clearance of lipoplexes lacking PEG after four intravenous administrations in immunocompetent Balb/c mice bearing 4T1 tumors. Tumor-bearing mice were administered four doses of lipoplexes via tail vein injection (one dose every three days), and blood samples were collected after each injection as described above. Consistent with our previous studies in immunocompetent mice, lipoplexes were cleared rapidly from the plasma (FIG. 8).

Clearance of the first dose was somewhat slower than subsequent doses, and plasmid was detected at very low levels in the plasma for 24 h after the initial dose, in contrast to subsequent doses (FIG. 8). The rapid blood clearance after each injection makes it difficult to determine if clearance of subsequent doses is accelerated, however the prolonged circulation of a small fraction of plasmid administered with the first dose suggests that the initial treatment does alter the pharmacokinetics in subsequent doses. We point out that the majority of the injected dose is cleared almost immediately (within 5 min), consistent with other pharmacokinetic studies of lipoplexes. But our data indicate that clearance of the initial dose is slightly reduced as compared to subsequent doses.

### Tumor Accumulation and Expression

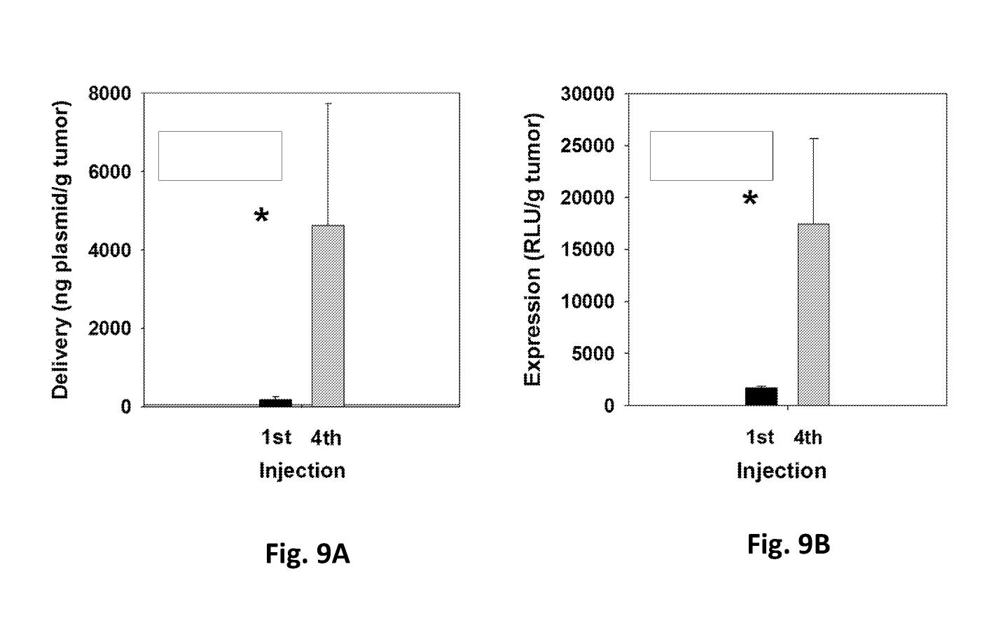

Our dosing protocol involved four doses administered at three-day intervals. Because the lipoplexes are rapidly cleared from the blood, there should be minimal potential for any dose to contribute to additional organ accumulation in the subsequent dosing (i.e., three days after injection). Accordingly, each injection of lipoplexes would be expected to result in similar accumulation in the tumor. In fact, considering the prolonged circulation of a small portion of lipoplexes from the first dose, one might expect that subsequent doses would contribute less to tumor accumulation. Furthermore, any plasmid that accumulates in the tumor (or any organ) would be expected to be degraded in and/or cleared from that tissue, and therefore we predicted that plasmid levels in the tumor after the fourth injection could only be a maximum of four-fold that observed after a single dose. However, our data indicate that both plasmid levels (26-fold) and luciferase expression (10-fold) in the tumor are enhanced by more than four-fold after the fourth dose as compared to that after a single administration (FIGS. 9A and 9B). The lower enhancement of expression as compared to plasmid levels may be due to plasmid accumulated in the tumor that may still be in the process of being expressed, e.g., has yet to be internalized, dissociated from the lipid carrier, or gain access to the nucleus. This same phenomenon could potentially explain why luciferase expression is enhanced by more than four-fold after a series of four injections, i.e., some plasmids delivered after a single injection have yet to be expressed at 24 h, but may be available for expression at later timepoints. While such an effect could potentially account for the boost in luciferase activity, this explanation is not applicable to the 26-fold enhancement in plasmid accumulation that we observe.

### Accumulation and Expression in Organs

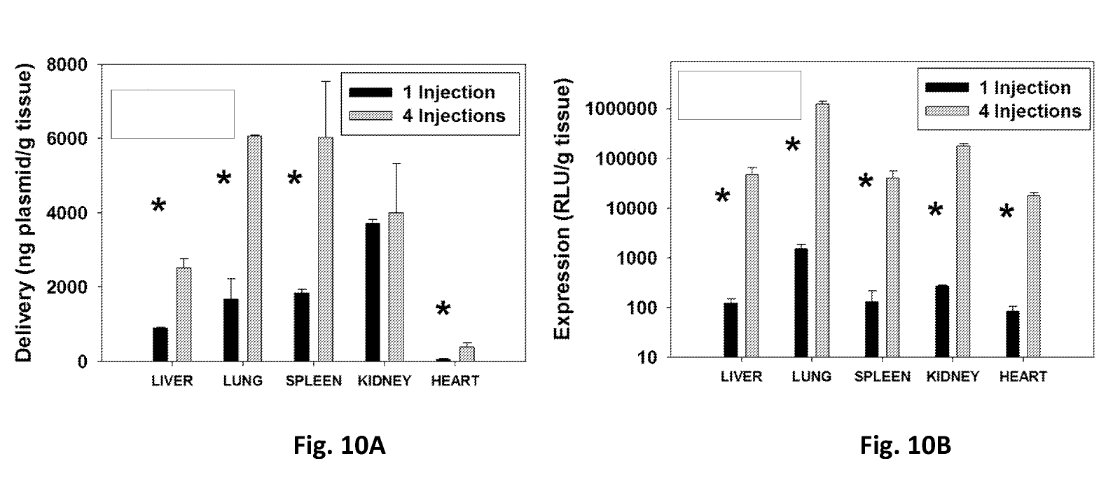

The effect of repetitive administration on the accumulation of plasmid in different organs was consistent with the expectation that enhancement after four administrations would be somewhat less than four-fold that observed after a single injection. The enhancement in plasmid levels in the liver, lung, spleen, and kidney ranged from 1.1- to 3.6-fold, with the heart exhibiting much lower levels that were enhanced 7.1-fold, as compared to a single injection (FIG. 10A). Although expression often does not correlate strongly with plasmid levels, luciferase activity was enhanced by greater than two orders of magnitude in each of the organs, varying between 210- and 815-fold as compared to that seen after a single injection (FIG. 10B). It is surprising that the modest increases in plasmid accumulation after four doses resulted in such dramatic increases in expression in each organ. This effect is especially surprising considering that the exact opposite trend was observed in the tumor, i.e., plasmid levels were enhanced to a greater extent than expression. Although we cannot reach definitive conclusions regarding the mechanisms involved, these data show that the process by which plasmid is internalized and expressed is fundamentally different in the tumor as compared to other organs.

### Liver Toxicity

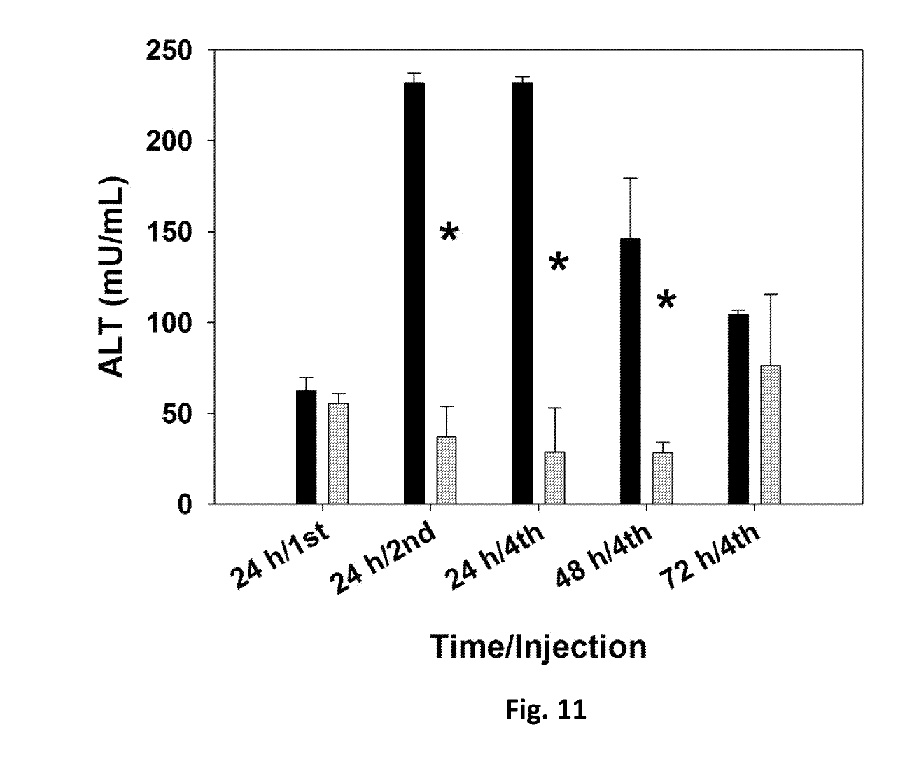

Liver toxicity was assessed by measuring the levels of alanine aminotransferase (ALT) in the blood 24 h after the first, second, and fourth injection. In addition, blood samples were also collected at 48 h and 72 h after a fourth injection to monitor the ability of the liver to recover from repetitive injections. A separate set of tumor-bearing mice was also administered PBS as a control to determine the extent to which ALT levels are altered in response to repetitive injections. As shown in FIG. 11, ALT levels 24 h after a single dose of lipoplexes were comparable to that exhibited by mice administered PBS. However, ALT levels were elevated by almost four-fold 24 h after the second and fourth dose, as compared to that observed after a single dose of lipoplexes. ALT levels gradually receded after the fourth dose eventually reaching levels that were elevated less than two-fold after 72 h, as compared to that seen 24 h after a single injection of lipoplexes (FIG. 11).

### In Vivo Imaging

Luciferase expression in live mice was imaged 24 h and 72 h after the first three injections, and 24 h after the fourth injection. FIG. 12 shows representative images at each time point from three different mice. The most striking result is the wide distribution of expression 24 h after each injection as compared to expression 72 h after each injection which is largely confined to the tumor. It is important to remember that the imaging of luciferase expression is highly depth-dependent, and therefore images that depict luciferase activity that is limited to the tumor should not be interpreted as evidence that expression occurs only in the tumor. Furthermore, organs were only extracted 24 h after the first and fourth injection, therefore reliable quantification of plasmid levels and luciferase expression at timepoints where imaging depicts expression primarily localized to the tumor (i.e., 72 h after each injection) was not possible. Attempts to quantitatively evaluate images from these experiments did not yield consistent results. However, successive images in each mouse show a general trend of progressively increasing luciferase expression in tumors after each dose of lipoplexes.

A recent study by Lindberg et al. reported that repeat administration of lipoplexes was only able to regain (i.e., not increase) expression levels observed after an initial dose (Lindberg M F, et al. Efficient in vivo transfection and safety profile of a CpG-free and codon optimized luciferase plasmid using a cationic lipophosphoramidate in a multiple intravenous administration procedure. Biomaterials. 2015; 59:1-11). These authors clearly demonstrated that a refractory period of several weeks was required between administrations in order to achieve transgene expression comparable to the initial dose, consistent with other studies. Our results differ sharply from these observations, and suggest that delivery is increased beyond that seen after the initial administration, and this was achieved by successive injections that were just three days apart. The refractory period observed in previous studies is thought to be closely related to immunostimulatory effects of both the nucleic acid and the delivery vehicle. As described above, we have utilized a plasmid with minimal CpG sequences, avoided the use of PEGylation, and employed naturally-occurring lipids, and these factors appear to be responsible for the lack of refractory period observed in our study.

Curiously, the enhancement of both plasmid accumulation and expression after repetitive administration showed opposite trends, i.e., the increase in plasmid accumulation exceeded that of expression in the tumor whereas the enhancement in plasmid expression greatly exceeded that of accumulation in the other organs. These distinctly different trends suggest that the timing of plasmid delivery relative to expression is fundamentally different in tumors as compared to other organs. More specifically, plasmid levels in the tumor at 24 h were >5-fold lower than that in the liver, lung, spleen or kidney, yet luciferase activity in the tumor was higher than any of these organs. Moreover, luciferase activity at 24 h was 11% higher in the tumor than in the lung, despite plasmid levels that were 90% lower! These results indicate that plasmid delivered to the tumor is more readily available for expression, in agreement with previous findings. This is consistent with the lower degree of expression enhancement in the tumor (10-fold) after repetitive injections as compared to that observed in the other organs (>200-fold) due to more rapid expression of delivered plasmids in the tumor at 24 h. This effect could potentially be due to the greater rate of cell division in tumors that allow plasmids to more readily access the nucleus as compared to cells associated with other tissues that divide more slowly. According to this hypothesis, plasmid delivered to non-tumor cells exhibit delayed expression due to reduced rates of cell division, and this contributes to the much larger enhancement of expression observed at later times (i.e., after 4 injections). Considering that the lifetime of free plasmids in the cytoplasm is only 50-90 minutes, the rate of lipoplex dissociation likely plays a role in maintaining plasmid integrity prior to mitosis.

In addition to the very different trends in luciferase activity noted above, repetitive administration was able to enhance plasmid delivery to the tumor to a much greater extent than in other organs. This curious result is consistent with the enhanced delivery to tumors observed in previous studies employing repetitive injections, but the precise mechanism responsible for this effect is unclear. Considering that the vasculature of tumors, in contrast to established organs, is rapidly changing, it is possible that particle deposition alters tumor vascularization. Such an effect has recently been described by Sabnani et al. (Liposome promotion of tumor growth is associated with angiogenesis and inhibition of antitumor immune responses. Nanomedicine: nanotechnology, biology, and medicine. 2015; 11:259-62) and earlier studies have demonstrated that particulate delivery systems primarily deposit on the tumor vasculature. Therefore, it is possible that particle deposition selectively stimulates greater vascularization in the tumor, which thereby enhances delivery via subsequent injections.

The images in FIG. 12 show a broad initial distribution throughout the body after 24 h that is predominantly confined to the tumor 72 h after each injection. Similar images have been generated after administration of fluorescently-labelled macromolecules, and the localization to the tumor in later images has been presented as evidence for reduced lymphatic clearance according to the enhanced permeation and retention effect. But it is important to realize that our images are fundamentally different because they depict expression of a delivered plasmid by recipient cells as opposed to distribution of the administered particles. As discussed above, previous studies have shown that efficient expression of plasmid requires cell division, and thus our images depict the presence of dividing cells throughout the body. This finding is consistent with studies showing that lipoplexes administered intravenously predominantly transfect the vascular endothelium. However, the images taken 72 h after each injection indicate that luciferase expression as detected by the IVIS imaging system is dramatically reduced in the majority of the body. The depth dependence of luminescence detection complicates interpretations of the images in FIG. 12, but these findings suggest that a large fraction of the luciferase expression present in tissues at 24 h is not observed at 72 h. Furthermore, this effect was clearly evident after each of the first three injections, and we speculate that transfected cells of the rapidly dividing endothelial layer are routinely sloughed off, resulting in the apparent disappearance of expression after 72 h. Although quantification of the extent to which plasmid accumulates in non-vascular tissues after repetitive injection would require more sophisticated experimentation, we hypothesize that characterization at later time points (i.e., ≥72 h) might provide results that more accurately reflect delivery to cells outside of the vasculature. The fact that the images do not depict dramatic reductions in luciferase activity within the tumors suggest that lipoplexes were able to more readily access longer-lived cells within the tumor, consistent with the altered vasculature associated with tumors.

In conclusion, these data demonstrate that our lipoplex formulation is rapidly cleared from the plasma, and that the initial injection persists for longer than subsequent injections, albeit at very low levels. Plasmid levels in the tumor after a single injection were relatively low compared to other organs, but accumulation was comparable on a per gram tissue basis after four injections administered at three-day intervals. In contrast, luciferase expression was higher in the tumor 24 h after the initial injection, but lower than lung, liver, and spleen 24 h after the fourth injection. Liver enzyme (ALT) levels were slightly elevated (2× that of saline) after the first injection, and became more highly elevated after subsequent administrations. In vivo imaging revealed a wide distribution of expression throughout the body 24 h after each injection that was predominantly confined to the tumor after 72 h. Most importantly, these results demonstrate that plasmid levels and reporter gene expression in the tumor are greatly enhanced after repetitive administration. The observation that delivery is enhanced by much greater than four-fold after four injections suggests that an administration of lipoplexes may preferentially affect the tumor environment in a way that promotes tumor uptake and/or retention of subsequent doses.

### Example 3

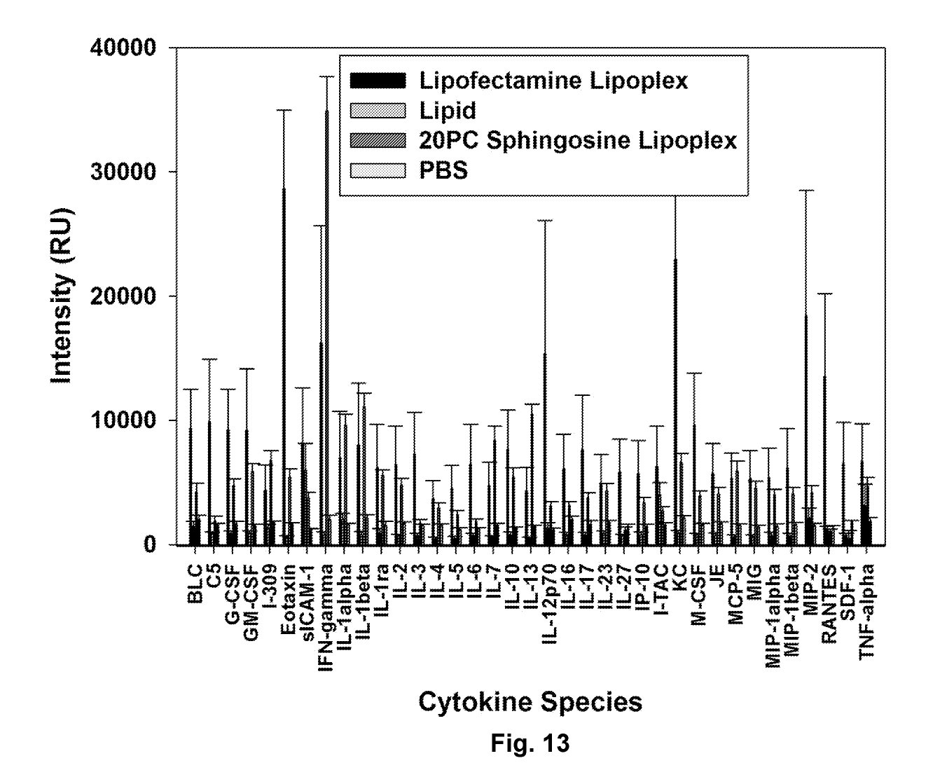

To evaluate the toxicity of lipoplexes of this disclosure, the inventors compared the cytokine response to the lipid formulation as compared to lipofectamine and phosphate buffered saline (PBS). Cytokine responses were evaluated by a commercial kit (Mouse Cytokine Panel from R&D systems. The levels of all the cytokines were measured in the blood of test mice at 24 h after IV administration of the lipoplex formulation of this disclosure (FIG. 13). The lipids and test plasmid were combined in water, after 15-20 minutes tonicity modulators (sucrose or hydroxyl ethyl starch), were added, and the resulting lipoplexes were injected into the tail vein of each mouse. These data show that the lipid formulation of these lipoplexes elicits a minimal cytokine response, and therefore elicits a minimal toxic response following administration.

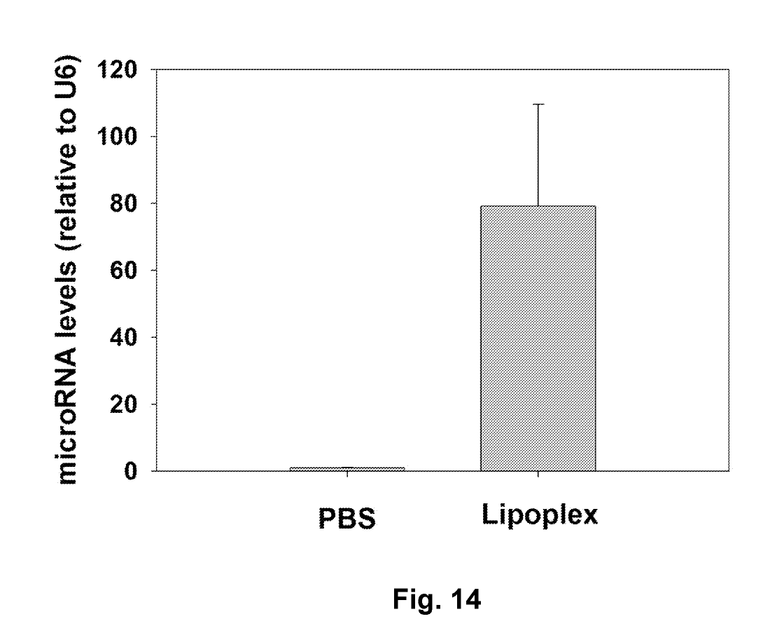

Tumors from the test animals were removed and analyzed. Quantitative PCR was used to measure miRNA levels encoded by the plasmid delivered by the administered lipoplex (FIG. 14). These data showed that plasmid expression is enhanced 80-fold in HEY tumors that are deficient in miR200c (p<0.01). This high expression could be from a small fraction of tumor cells. This demonstrates that miRNA is expressed following administration in these lipoplex formulations similar to the luciferase promoter described above.

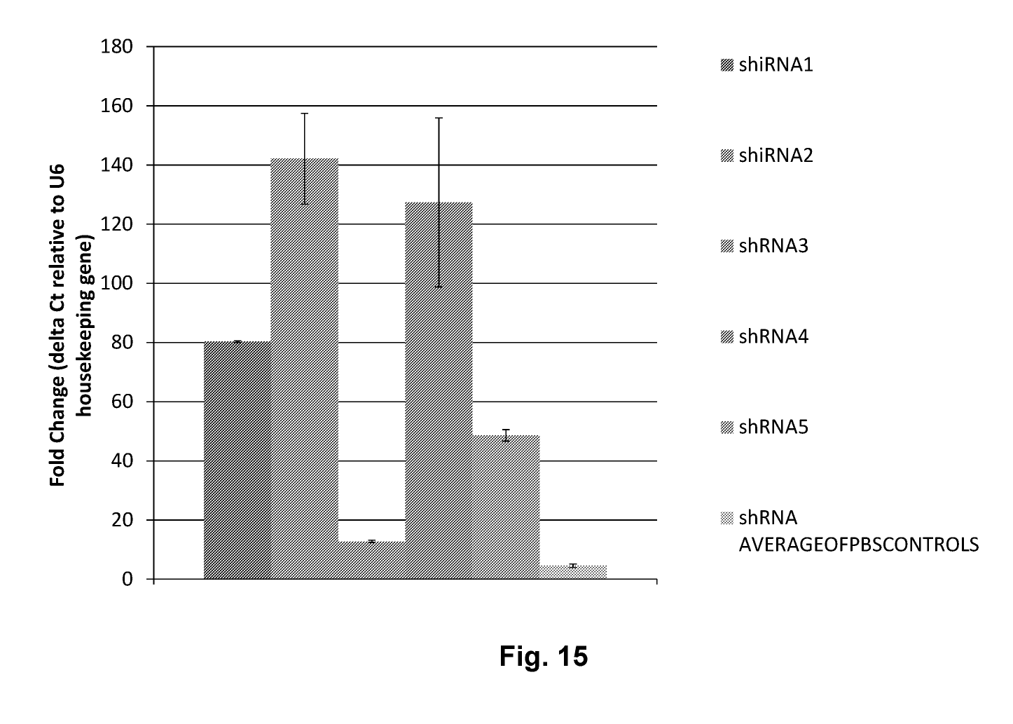

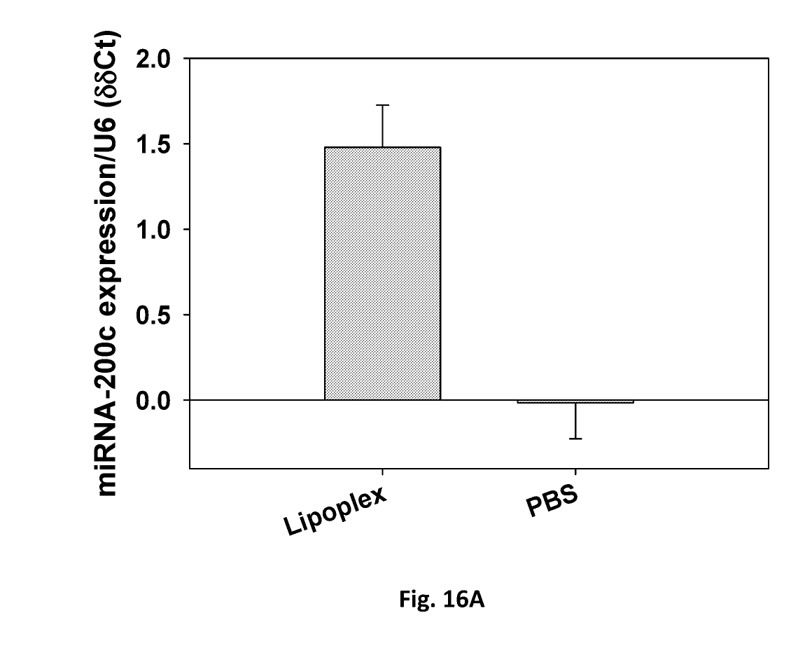

The expression construct in these lipoplexes encoded RNA sequences that silence PD-L1. FIG. 15 shows that 5 days after IV administration of the lipoplexes the messenger RNA for PD-L1 (as measured by PCR probes) is reduced by 95% in the tumor, compared to tumors from animals that were treated with PBS.

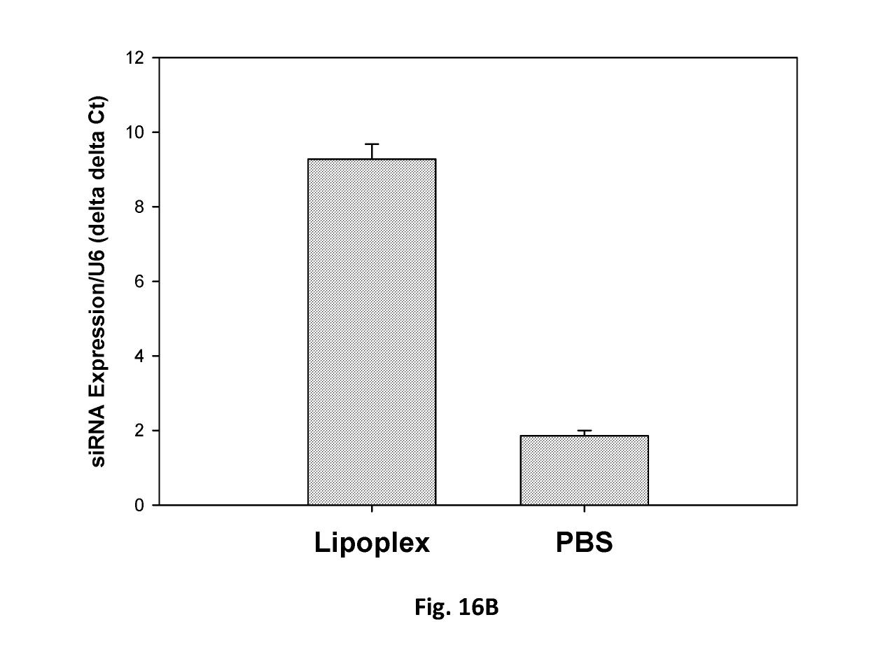

In a similar experiment evaluating transfection and expression in an in vitro model, exosomes harvested from cells transfected with lipoplexes of this disclosure contain the expressed miRNA. FIG. 16A shows miRNA-200c expression after transfection in the lipoplexes of this disclosure versus exposure to the cells in PBS. Similarly, FIG. 16B shows the expression of siRNA against PD-L1 after transfection in the lipoplexes of this disclosure versus exposure to the cells in PBS. These data demonstrated that the lipoplexes allowed uptake into the endogenous distribution pathway in these cells.

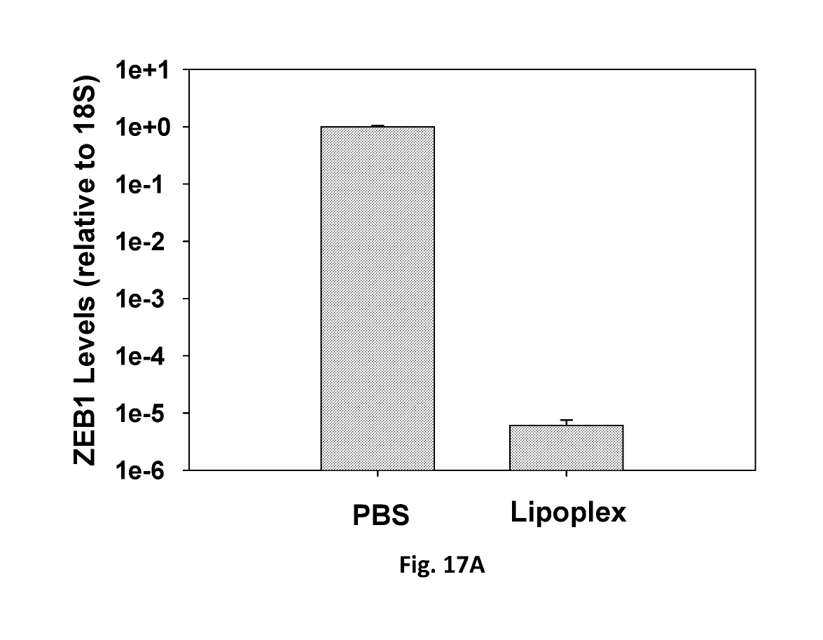

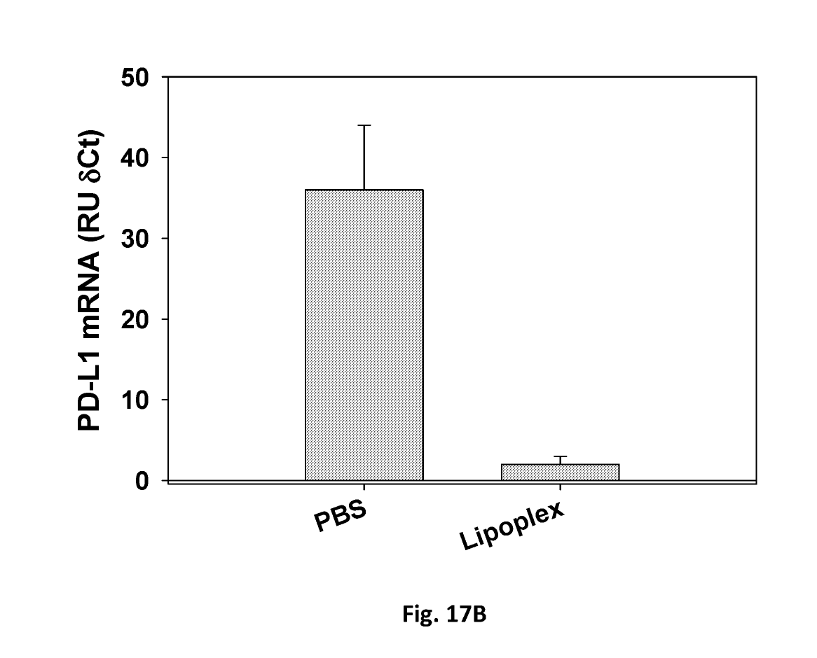

FIG. 17A shows that the ZEB1 gene (targeted by the miR200c) was nearly completely silenced in these cells transfected with the lipoplexes of this disclosure. Similarly, FIG. 17B shows that PD-L1 mRNA (targeted by the anti-PD-L1 siRNA) expression was substantially knocked down nearly completely silenced in these cells transfected with the lipoplexes of this disclosure. These efficacy results are also due to distribution via the exosomal pathway in the tested cells because virtually every cell in the tumor has it's expression of targeted messenger RNA (either ZEB1 or PD-L1) essentially shut down.

The foregoing examples of the present disclosure have been presented for purposes of illustration and description. Furthermore, these examples are not intended to limit the disclosure to the form disclosed herein. Consequently, variations and modifications commensurate with the teachings of the description of the disclosure, and the skill or knowledge of the relevant art, are within the scope of the present disclosure. The specific embodiments described in the examples provided herein are intended to further explain the best mode known for practicing the disclosure and to enable others skilled in the art to utilize the disclosure in such, or other, embodiments and with various modifications required by the particular applications or uses of the present disclosure. It is intended that the appended claims be construed to include alternative embodiments to the extent permitted by the prior art.

1. A lipoplex formulation comprising:
a. at least one cationic amphiphile,
b. at least one C₁₈₋₃₀ saturated fatty acid, and
c. at least one nucleic acid.

2. The lipoplex formulation of claim 1, further comprising cholesterol.

3. The lipoplex formulation of claim 2, wherein at least a portion of the cholesterol is conjugated to a ligand.

4. The lipoplex formulation of claim 3, wherein the ligand is a peptide ligand selected from the group consisting of cyclic RGD (cRGD), internalizing RGD (iRGD), polyarginines (RRRRRRRRR; SEQ ID NO:1), TAT (GRKKRRQRRRPPQ; SEQ ID NO:2), M918 (MVTVLFRRLRIRRACGPPRVRV; SEQ ID NO:3), Penetratin (RQIKIWFQNRRMKWKK; SEQ ID NO:4), TP10 (AGYLLGKINLKALAALAKKIL; SEQ ID NO:5), MPG (GALFLGFLGAAGSTMGAWSQPKKKRKV; SEQ ID NO:6), KALA (WEAKLAKALAKALAKHLAKALAKALKACEA; SEQ ID NO:7), ppTG1 (GLFKALLKLLKSLWKLLLKA; SEQ ID NO:8), MPGΔNLS (GALFLGFLGAAGSTMGAWSQPKKKRKV; SEQ ID NO:9), MPGα (GALFLAFLAAALSLMGLWSQPKKKRKV; SEQ ID NO:10), Chol-R9 (Cholesteryl-RRRRRRRRR; SEQ ID NO:11), CADY (Ac-GLWRALWRLLRSLWRLLWRA (SEQ ID NO:12)-cysteamide), and LMWP (VSRRRRRRGGRRRR; SEQ ID NO:13).

5. The lipoplex formulation of claim 2, wherein the cholesterol content is greater than 5% by weight.

6. The lipoplex formulation of claim 2, wherein the cholesterol content is between about 10% by weight and about 30% by weight.

7. The lipoplex formulation of claim 1, wherein the C₁₈₋₃₀ saturated fatty acid content is greater than 15% by weight.

8. The lipoplex formulation of claim 1, wherein the C₁₈₋₃₀ saturated fatty acid content is between about 20% by weight and about 60% by weight.

9. The lipoplex formulation of claim 1, wherein the saturated fatty acids used in the lipoplexes is predominately C₁₈₋₂₄ saturated fatty acids.

10. The lipoplex formulation of claim 1, wherein the a cationic amphiphile content is greater than 1% by weight.

11. The lipoplex formulation of claim 1, wherein the cationic amphiphile content is between about 5% by weight and about 30% by weight.

12. The lipoplex formulation of claim 1, wherein the cationic amphiphile content includes one or more of sphingosine, sphinganine, dimethylsphingosine, phytosphingosine, and stearylamine.

13. The lipoplex formulation of claim 1, having a charge ratio between 0.1 and 20.0.

14. The lipoplex formulation of claim 1, having a charge ratio between 0.25 and 4.0.

15. The lipoplex formulation of claim 1, having a charge ratio between 0.25 and 1.0.

16. The lipoplex formulation of claim 1, that is substantially-free of PEG, or derivatives thereof.

17. The lipoplex formulation of claim 1, that is completely-free of PEG, or derivatives thereof.

18. A pharmaceutical composition comprising the lipoplex formulation of claim 1, and a pharmaceutically acceptable excipient.

19. A kit comprising at least one component selected from a cationic amphiphile, at least one C₁₈₋₃₀ saturated fatty acid, and instructions for forming a lipoplex containing a user-supplied polyanionic therapeutic.

20. The kit of claim 19, further comprising cholesterol.
